(19) United States
(12) Patent Application Publication
(10) Pub. No.: US 2026/0137365 A1
(43) Pub. Date: May 21, 2026

(54) Localizing Inserted Medical Device and External Acoustic Source Using One-Way Time-of-Flight Measurements and Graphical Feedback

(71) Applicant: AVVIO Medical, Inc., San Francisco, CA (US)

(72) Inventors: Yuri A. Pishchalnikov, Rohnert Park, CA (US); Rohit Girotra, San Francisco, CA (US); Michal Jenco, San Carlos, CA (US); Matthew Hopcroft, San Francisco, CA (US)

(21) Appl. No.: 19/450,921

(22) Filed: Jan. 16, 2026

Related U.S. Application Data
(63) Continuation-in-part of application No. 18/645,970, filed on Apr. 25, 2024.
(60) Provisional application No. 63/498,297, filed on Apr. 26, 2023.

(51) Int. Cl. A61B 8/08; A61B 8/00; A61B 17/00; A61B 17/34

(52) U.S. Cl. CPC A61B 8/0841; A61B 8/461; A61B 17/3403; A61B 2017/00106; A61B 2017/3407; A61B 2017/3413; A61B 2560/0462; A61B 2562/0204

(57) ABSTRACT

An external acoustic source and a medical device inserted into a mammal are localized with respect to each other using one-way time-of-flight (ToF) measurements of acoustic signals sent from one or more transducer elements of a transducer in the external acoustic source to one or more acoustic sensors in the medical device. The ToF measurements are used to determine an alignment state of the transducer relative to the acoustic sensor(s). Graphical feedback data indicating the alignment state are generated and displayed to assist with achieving alignment of the transducer relative to the acoustic sensor(s).

### CROSS-REFERENCE TO RELATED APPLICATIONS

This application is a continuation-in-part of U.S. patent application Ser. No. 18/645,970, filed on Apr. 25, 2024, titled “Medical Device With Acoustic Sensor(s) and Method for Localizing Medical Device and Acoustic Source,” which claims priority to U.S. Provisional Application No. 63/498,297, filed on Apr. 26, 2023, titled “System and Method for Positioning a Treatment Apparatus With Respect to a Catheter.” Each of the foregoing applications is hereby incorporated by reference.

### TECHNICAL FIELD

This application relates generally to a medical device having one or more acoustic sensors and localizing an acoustic source and such a medical device with respect to each other.

### BACKGROUND

Many treatment procedures require positioning and monitoring the location of a catheter in a vessel. Although X-ray imaging can allow visualization of the catheter, alternative approaches without the use of ionizing radiation are preferable. Ultrasound scanners can provide nonionizing visualization, but the image resolution can be challenging, making it difficult to reliably identify and monitor the position of the catheter. For example, image resolution is better at higher frequencies which has poor penetration depth, thus making ultrasound imaging difficult at larger depths. It would be desirable to improve the ability to localize and monitor the position of the catheter without the use of ionizing radiation.

For many treatment procedures it would be desirable to use the position of the catheter as a marker, e.g., for the delivery of electromagnetic or acoustic energy into a specific region in a human body. The delivery of acoustic energy to the target is typically associated with various challenges. One challenge is alignment of the ultrasound beam on the target, which is also affected by diffraction and deflection of acoustic waves passing through different layers of tissue along the acoustic path to the target. Another challenge is acoustic coupling between the acoustic source and the skin of a patient, as well as ensuing an acoustic window in a patient body for efficient passage of acoustic waves to the target.

### SUMMARY

Example embodiments described herein have innovative features, no single one of which is indispensable or solely responsible for their desirable attributes. The following description and drawings set forth certain illustrative implementations of the disclosure in detail, which are indicative of several exemplary ways in which the various principles of the disclosure may be carried out. The illustrative examples, however, are not exhaustive of the many possible embodiments of the disclosure. Without limiting the scope of the claims, some of the advantageous features will now be summarized. Other objects, advantages, and novel features of the disclosure will be set forth in the following detailed description of the disclosure when considered in conjunction with the drawings, which are intended to illustrate, not limit, the invention.

An aspect of the invention is directed to a method for localizing an acoustic source and a medical device with respect to each other, the method comprising: a. introducing the medical device into a mammal, the medical device including an acoustic sensor; b. acoustically coupling the acoustic source to the mammal at a position that corresponds to a target location of the medical device, the acoustic source comprising at least a first source transducer element and a second source transducer element; c. sequentially producing acoustic signals with at least the first and second transducers; d. receiving the acoustic signals with the acoustic sensor on or in the medical device, the acoustic sensor in electrical or wireless communication with a detector; e. determining, with the detector, one-way time-of-flights (ToFs) of the acoustic signals transmitted at least from the first source transducer element to the acoustic sensor and from the second source transducer element to the acoustic sensor; f. determining, with the detector, an alignment state of the acoustic source with respect to the medical device based, at least in part, on the one-way ToFs of the acoustic signals; g. generating, with the detector, graphical feedback data indicating the alignment state of the acoustic source with respect to the medical device; and h. displaying, on a display in communication with the detector, the graphical feedback data.

In one or more embodiments, the method further comprises determining, with the detector and using the one-way ToFs of the acoustic signals, an axial position of the acoustic source relative to the medical device. In one or more embodiments, the method further comprises determining, with the detector, (a) a first distance between the first transducer element and the acoustic sensor using a first one-way ToF of the acoustic signals transmitted from the first source transducer element and the acoustic sensor (b) a second distance between the second transducer element and the acoustic sensor using a second one-way ToF of the acoustic signals transmitted from the second source transducer element and the acoustic sensor; determining, with the detector, a radial distance between the acoustic source and the acoustic sensor using the first and second distances, the radial distance measured along a radial axis that is orthogonal to a reference axis that extends along a surface of the first and second source transducer elements, wherein an intersection of the radial axis and the reference axis corresponds to the axial position of the acoustic source relative to the medical device.

In one or more embodiments, the alignment state is an axial misalignment of the acoustic source with respect to the medical device in a first direction along a reference axis that extends along a surface of the first and second source transducer elements, and the graphical feedback data indicates a second direction to move the acoustic source along or parallel to the reference axis, the second direction opposite to the first direction. In one or more embodiments, the method further comprises: i. moving the acoustic source along or parallel to the reference axis; and j. repeating steps c-h in a loop so as to update the graphical feedback data on the display in real time while moving the acoustic source in step i.

In one or more embodiments, the method further comprises determining, with the detector and using the one-way ToFs of the acoustic signals, that the acoustic source is axially aligned with the medical device; and after determining that the acoustic source is axially aligned with the medical device: changing the alignment state to an axial alignment of the acoustic source with respect to the medical device; and updating the graphical feedback data to indicate the axial alignment of the acoustic source with respect to the medical device.

In one or more embodiments, the acoustic sensor is disposed closer to a distal end of the medical device than to a proximal end of the medical device, such that the alignment state corresponds to relative positions of the acoustic source and the distal end of the medical device.

Another aspect of the invention is directed to a system comprising: a medical device having a shaft configured to be inserted into a mammal, the shaft including an acoustic sensor; an acoustic source including a housing and a transducer having a plurality of transducer elements including first and second source transducer elements, the acoustic source configured to be acoustically coupled to an external surface of the mammal; a controller in communication with the acoustic source, the controller configured to cause the at least first and second source transducer elements to sequentially produce acoustic signals; a detector in communication with the acoustic sensor, the detector including a processor and non-volatile memory coupled to the processor, the non-volatile memory storing computer-readable instructions that, when executed by the processor, cause the processor to: a. determine one-way time-of-flights (ToFs) of the acoustic signals transmitted at least from the first source transducer element to the acoustic sensor and from the second source transducer element to the acoustic sensor; b. determine an alignment state of the acoustic source with respect to the medical device based, at least in part, on the one-way ToFs of the acoustic signals; and c. generate graphical feedback data indicating the alignment state of the acoustic source with respect to the medical device; and a display in communication with the detector and configured to display the graphical feedback data.

In one or more embodiments, the computer-readable instructions, when executed by the processor, further cause the processor to determine, using the one-way ToFs of the acoustic signals, an axial position of the acoustic source relative to the medical device. In one or more embodiments, the computer-readable instructions, when executed by the processor, further cause the processor to: calculate (a) a first distance between the first transducer element and the acoustic sensor using a first one-way ToF of the acoustic signals transmitted from the first source transducer element and the acoustic sensor (b) a second distance between the second transducer element and the acoustic sensor using a second one-way ToF of the acoustic signals transmitted from the second source transducer element and the acoustic sensor; and determine a radial distance between the acoustic source and the acoustic sensor using the first and second distances, the radial distance measured along a radial axis that is orthogonal to a reference axis that extends along a surface of the first and second source transducer elements, wherein an intersection of the radial axis and the reference axis corresponds to the axial position of the acoustic source relative to the medical device. In one or more embodiments, the alignment state is an axial misalignment of the acoustic source with respect to the medical device in a first direction along a reference axis that extends along a surface of the first and second source transducer elements, and the graphical feedback data indicates a second direction to move the acoustic source along or parallel to the reference axis, the second direction opposite to the first direction.

In one or more embodiments, the acoustic sensor is a first acoustic sensor, the shaft includes a second acoustic sensor that is axially displaced from the first acoustic sensor along the shaft, the one-way ToFs are determined at least from the first source transducer element to each of the first and second acoustic sensors and from the second source transducer element to each of the first and second acoustic sensors, and the computer-readable instructions, when executed by the processor, further cause the processor to: determine an axial alignment state of the acoustic source relative to each of the first and second acoustic sensors, the axial alignment state determined with respect to a first reference axis that extends along a surface of the first and second source transducer elements; determine a first rotational alignment state of the medical device relative to the acoustic source, the first rotational alignment state representing a rotational orientation of the acoustic source about the first reference axis; and determine a second rotational alignment state of the acoustic source relative to the medical device, the second rotational alignment state representing a rotational orientation of the acoustic source about a second reference axis that is orthogonal to the first reference axis.

In one or more embodiments, the computer-readable instructions, when executed by the processor, further cause the processor to: calculate, using the one-way ToFs of the acoustic signals sent from the first and second transducer elements to the first acoustic sensor, an axial position of the first acoustic sensor relative to the acoustic source; calculate, using the one-way ToFs of the acoustic signals sent from the first and second transducer elements to the second acoustic sensor, an axial position of the second acoustic sensor relative to the acoustic source; compare the axial positions of the first and second acoustic sensors relative to the acoustic source with a center of the transducer; and determine that the first and second acoustic sensors are axially aligned with the transducer when the axial positions of the first and second acoustic sensors are within a predetermined range of the center of the transducer, wherein the graphical feedback data indicates an axial alignment of the transducer relative to the first and second acoustic sensors when the axial positions of the first and second acoustic sensors are within the predetermined range of the center of the transducer.

In one or more embodiments, the computer-readable instructions, when executed by the processor, further cause the processor to: determine that first acoustic sensor is not axially aligned with the transducer when the axial position of the first acoustic sensor is outside of the predetermined range of the center of the transducer; and determine that second acoustic sensor is not axially aligned with the transducer when the axial position of the second acoustic sensor is outside of the predetermined range of the center of the transducer, wherein the graphical feedback data indicates an axial misalignment of the transducer relative to the first acoustic sensor and/or the second acoustic sensor when the axial position of the first acoustic sensor is outside of the predetermined range of the center of the transducer and/or the axial position of the second acoustic sensor is outside of the predetermined range of the center of the transducer, respectively. In one or more embodiments, the graphical feedback data further indicates an axial direction to move the acoustic source to achieve the axial alignment of the transducer relative to the first and second acoustic sensors.

In one or more embodiments, the computer-readable instructions, when executed by the processor, further cause the processor to: calculate (a) a first distance between the first transducer element and the first acoustic sensor using a one-way ToF of the acoustic signals transmitted from the first source transducer element and the first acoustic sensor (b) a second distance between the second transducer element and the first acoustic sensor using a one-way ToF of the acoustic signals transmitted from the second source transducer element and the first acoustic sensor; calculate (c) a third distance between the first transducer element and the second acoustic sensor using a one-way ToF of the acoustic signals transmitted from the first source transducer element and the second acoustic sensor (d) a fourth distance between the second transducer element and the first acoustic sensor using a second one-way ToF of the acoustic signals transmitted from the second source transducer element and the second acoustic sensor; determine a first radial distance between the acoustic source and the first acoustic sensor using the first and second distances, the first radial distance measured along a first radial axis that is orthogonal to the first reference axis and that is parallel to the second reference axis; and determine a second radial distance between the acoustic source and the second acoustic sensor using the third and fourth distances, the second radial distance measured along a second radial axis that is orthogonal to the first reference axis and that is parallel to the second reference axis, wherein an intersection of the first radial axis and the first reference axis corresponds to a first axial position of the first acoustic sensor relative to the transducer, an intersection of the second radial axis and the first reference axis corresponds to a second axial position of the first acoustic sensor relative to the transducer, and the graphical feedback data includes a representation of the first and second axial positions.

In one or more embodiments, the computer-readable instructions, when executed by the processor, further cause the processor to: compare the first and second radial distances; determine that the transducer is rotationally aligned, about the first reference axis, with the first and second acoustic sensors when the first and second radial distances are within a predetermined range of each other; and determine that the transducer is not rotationally aligned, about the first reference axis, with the first and second acoustic sensors when the first and second radial distances are outside of the predetermined range of each other, wherein the graphical feedback data indicates a rotational alignment of the transducer, about the first reference axis, with respect to the first and second acoustic sensors when the first and second radial distances are within a predetermined range of each other, and the graphical feedback data indicates a rotational misalignment of the transducer, about the first reference axis, with the first and second acoustic sensors when the first and second radial distances are outside of the predetermined range of each other.

In one or more embodiments, the graphical feedback data further indicates a rotational direction to rotate the acoustic source, about the first reference axis, to achieve the rotational alignment of the transducer, about the first reference axis, with respect to the first and second acoustic sensors.

In one or more embodiments, the computer-readable instructions, when executed by the processor, further cause the processor to: compare the first and second axial positions; determine that the transducer is rotationally aligned, about the second reference axis, with the first and second acoustic sensors when the first and second axial positions are within a predetermined range of each other; and determine that the transducer is not rotationally aligned, about the second reference axis, with the first and second acoustic sensors when the first and second axial positions are outside of the predetermined range of each other, wherein the graphical feedback data indicates that a rotational alignment of the transducer, about the second reference axis, with the first and second acoustic sensors when the first and second axial positions are within a predetermined range of each other, and the graphical feedback data indicates a rotational misalignment of the transducer, about the second reference axis, with the first and second acoustic sensors when the first and second axial positions are outside of the predetermined range of each other.

In one or more embodiments, the graphical feedback data further indicates a rotational direction to rotate the acoustic source, about the second reference axis, to achieve the rotational alignment of the transducer, about the second reference axis, with respect to the first and second acoustic sensors.

### BRIEF DESCRIPTION OF THE DRAWINGS

For a fuller understanding of the nature and advantages of the concepts disclosed herein, reference is made to the detailed description and the accompanying drawings.

FIG. 1 is a block diagram of a system for acoustically localizing an acoustic source with respect to a catheter according to an embodiment.

FIG. 2 is an isometric view of the catheter illustrated in FIG. 1 according to an embodiment.

FIG. 3A is an enlarged view of the distal end of the catheter illustrated in FIG. 2.

FIG. 3B is a top view of a piezoelectric film including two rectangular active sensing elements formed on the same substrate, with overlapping electrodes defining the sensitive regions and conductive leads connecting each element to its respective wire attachment area.

FIG. 3C illustrates a folded piezoelectric film configuration in which the ground electrode is located on both external surfaces and the signal electrode is enclosed between folded layers to provide EMI shielding and allow full circumferential wrapping around the catheter shaft without electrical shorting.

FIG. 3D shows an alternative piezoelectric film wrapping configuration forming a continuous 360° cylindrical sensing band around the catheter shaft to achieve omnidirectional acoustic sensitivity independent of catheter rotation or orientation.

FIG. 3E illustrates a piezoelectric film cut in a parallelogram shape and wrapped spirally or helically along the catheter shaft to increase the effective sensing area, enhance coupling with incident acoustic waves, and tune axial and circumferential sensitivity.

FIG. 3F shows a parallelogram-shaped piezoelectric film incorporating two active sensing elements on a single substrate, allowing improved spatial resolution and enhanced sensitivity for detecting acoustic waves along the catheter shaft.

FIG. 3G illustrates the piezoelectric film sensor of FIG. 3F wrapped around the catheter shaft, forming a dual-element configuration suitable for circumferential acoustic detection.

FIG. 3H is a cross-sectional view of the catheter shaft showing three piezoelectric film sensors positioned circumferentially around the shaft to provide independent detection of acoustic waves and spatial resolution of the catheter position.

FIG. 3I illustrates the unwrapped configuration of the piezoelectric film from FIG. 3H, showing three active sensing elements and a common ground formed on opposite sides of the film, each sensor connected by individual leads to separate wire attachment pads.

FIGS. 4A-C are a cross sections of the shaft of the catheter by a first plane according to different embodiments.

FIG. 5 is a cross section of the shaft of the catheter by a second plane according to an embodiment.

FIGS. 6A-D are cross sections of the shaft of the catheter by a third plane according to different embodiments.

FIG. 7 is a top view of the housing of the catheter illustrated in FIG. 2 with the cover removed.

FIG. 8 is a flow chart of a method for localizing an acoustic source and a catheter with respect to each other.

FIG. 9 illustrates an example localization method according to an embodiment.

FIG. 10 illustrates an example localization method according to another embodiment.

FIG. 11 is a flow chart of a method for localizing an acoustic source and a catheter with respect to each other according to another embodiment.

FIG. 12 shows an example coordinate system.

FIGS. 13A and 13B show example traces of two acoustic sensors on a catheter and their respective differential signals.

FIG. 14 shows example traces from two acoustic sensors on a catheter oriented along the acoustic axis of the acoustic source.

FIG. 15 is a block diagram of a system for acoustically localizing an acoustic source with respect to a catheter according to another embodiment.

FIG. 16 is a partially transparent side view of a guidewire according to an embodiment.

FIGS. 17A and 17B are cross sections of the guidewire illustrated in FIG. 16 according to different embodiment.

FIG. 18 is a partially transparent side view of a guidewire according to another embodiment.

FIGS. 19A-C are cross sections of the guidewire illustrated in FIG. 18 according to different embodiments.

FIG. 20 is a block diagram of a system for acoustically localizing an acoustic source with respect to a guidewire according to an embodiment.

FIG. 21 shows an example embodiment in which a medical device with multiple acoustic sensors is inserted through the ureter in the collecting system of the human kidney.

FIG. 22 is a flow chart of a method for localizing an acoustic source and a catheter with respect to each other according to one or more embodiments.

FIG. 23 shows an acoustic source and a catheter in a misaligned state according to one or more embodiments.

FIG. 24 shows an acoustic source and a catheter in an aligned state according to one or more embodiments.

FIG. 25 shows graphical feedback data representing axial misalignment of an acoustic source relative to a catheter according to one or more embodiments.

FIG. 26 shows graphical feedback data representing alignment of an acoustic source relative to a catheter according to one or more embodiments.

FIG. 27 shows an acoustic source and a catheter in a rotationally misaligned state according to one or more embodiments.

FIG. 28 shows graphical feedback data representing rotational misalignment of an acoustic source relative to a catheter according to one or more embodiments.

FIG. 29 shows an acoustic source and a catheter in a rotationally aligned state according to one or more embodiments.

FIG. 30 shows an acoustic source and a catheter in a rotationally misaligned state according to one or more embodiments.

FIG. 31 shows graphical feedback data representing rotational misalignment of an acoustic source relative to a catheter according to one or more embodiments.

FIG. 32 shows an acoustic source and a catheter in an axially misaligned state according to one or more embodiments.

FIG. 33 is a flow chart of step 2207 in FIG. 22 according to one or more embodiments.

FIG. 34A shows an acoustic source is coupled to the skin of a human mammal in the lower back region.

FIG. 34B is a perspective view of the acoustic source and its corresponding treatment region according to one or more embodiments.

FIG. 35 shows a front perspective view of the transducer as seen from the direction of the catheter according to one or more embodiments.

FIG. 36 shows a front perspective view of the transducer as seen from the direction of the catheter according to one or more embodiments.

FIG. 37 shows a front perspective view of the transducer as seen from the direction of the catheter according to one or more embodiments.

FIG. 38 shows a front perspective view of the transducer as seen from the direction of the catheter according to one or more embodiments.

FIG. 39 shows a front perspective view of the transducer as seen from the direction of the catheter according to one or more embodiments.

FIG. 40 shows a front perspective view of the transducer as seen from the direction of the catheter, with the transducer center aligned to the catheter center, according to one or more embodiments.

### DETAILED DESCRIPTION

A catheter includes a shaft having one or more acoustic sensors disposed on or in the shaft and at respective distance(s) from a distal end of the shaft. The acoustic sensor(s) can include a respective piezoelectric polymer film that is disposed about some or all of the circumference of an inner tube or of an outer tube of the shaft. The outer tube is disposed over the inner tube to cover the acoustic sensor(s). Leads and wires for the acoustic sensors can be disposed between the inner and outer tubes.

A guidewire includes a coil, a core, and having one or more acoustic sensors disposed on or in the guidewire at respective distance(s) from a distal end of the guidewire. The acoustic sensor(s) can include a respective piezoelectric polymer film that is disposed about some or all of the circumference of a protective coating of the coil or about some or all of the circumference of the core.

The acoustic sensor(s) can be used to localize the catheter or guidewire and an acoustic source with respect to each other, without imaging, after the catheter or guidewire is inserted or introduced into a mammal.

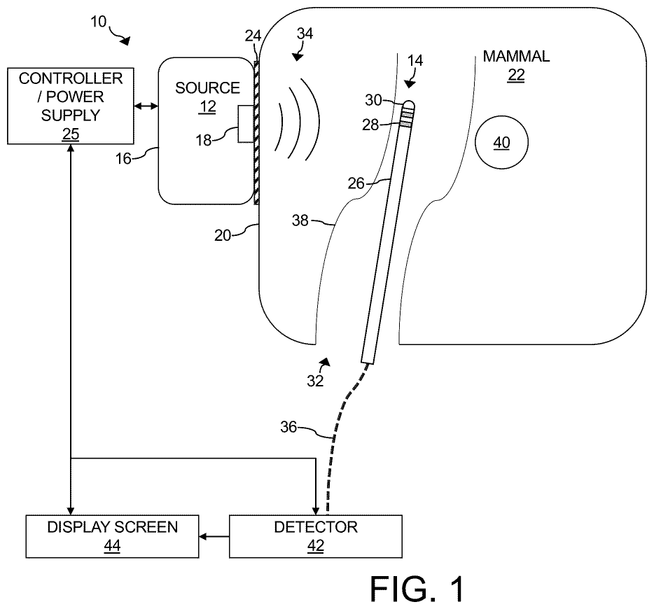

FIG. 1 is a block diagram of a system 10 for acoustically localizing an acoustic source 12 with respect to a catheter 14 according to an embodiment. The acoustic source 12 includes a housing 16 and one or more acoustic transducers 18 disposed in the housing 16. The acoustic source 12 can comprise an acoustic treatment head that can be configured to produce acoustic energy to perform therapy or a medical procedure. The acoustic source 12 can be placed on the skin 20 of a mammal 22 such as a human. An acoustically transmitting media 24, such as water, a water cushion, an acoustic coupling oil, and/or an acoustic coupling gel, can be disposed between (e.g., in direct physical contact with) the acoustic source 12 and the skin 20 to improve acoustic transmission. The acoustic source 12 can be powered by a power supply, an amplifier, and/or a controller 25 that is electrically coupled to the acoustic source 12.

The catheter 14 includes a shaft 26 and one or more acoustic sensors 28 disposed on or in the shaft 26. The acoustics sensor(s) 28 can be located at predetermined position(s) from a distal end of the shaft 26 and/or from a tip 30 at the distal end of the catheter 14. After the catheter 14 is introduced into the mammal 22 such as a through a natural or surgical opening 32, the acoustic source 12 and the catheter 14 can be localized and/or aligned with respect to each other using acoustic signals 34 produced by the acoustic source 12 and received by the acoustic sensor(s) 28 on the catheter 14. For example, the time-of-flight (ToF) and/or amplitude (e.g., maximum) of the acoustic signals 34 can be used to localize and/or align the acoustic source 12 and the catheter 14. The catheter 14 can be placed in an anatomical structure in the mammal 22, such as in an anatomical channel 38 (e.g., the urethra, the rectum, or a blood vessel), an internal organ, or another anatomical structure. The catheter 14 can be placed near a target volume 40 that may be the target of a therapeutic and/or a medical procedure. The catheter 14 can be used to introduce a guidewire, a tool, an acoustic enhancer (e.g., engineered microbubbles), fluids, and/or a therapeutic substance to or near the target volume 40. For example, an acoustic enhancer can be used to facilitate breakage of calcifications, such as urinary stone(s) or kidney stone(s), at low pressure amplitudes by promoting localized cavitation. The target volume 40 can be located in the anatomical channel 38 in one or more embodiments.

The acoustic sensor(s) 28 can be electrically coupled (e.g., via a cable or wire(s) 36 (in general, cable)) to a detector 42 to detect and/or analyze the acoustic signals (e.g., acoustic signals 34) received by the acoustic sensor(s) 28, which have been converted by the acoustic sensor(s) 28 to electrical signals. Additionally or alternatively, the electrical signal data, representing the acoustic signals received by the acoustic sensor(s) 28, can be transmitted wirelessly to the detector 42. The wireless transmission can be performed using a local wireless protocol such as Bluetooth, a local wireless network such as WiFi, a wide area wireless network such as a cellular network, or another wireless transmission network or protocol.

The detector 42 can comprise a computer, a treatment console (e.g., a portion of a treatment console), a data acquisition board, an oscilloscope, or any other device to precondition and/or detect the acoustic signals received by the acoustic sensor(s) 28. In some embodiments, the detector 42 and the controller/power supply 25 can be combined, for example in a treatment console.

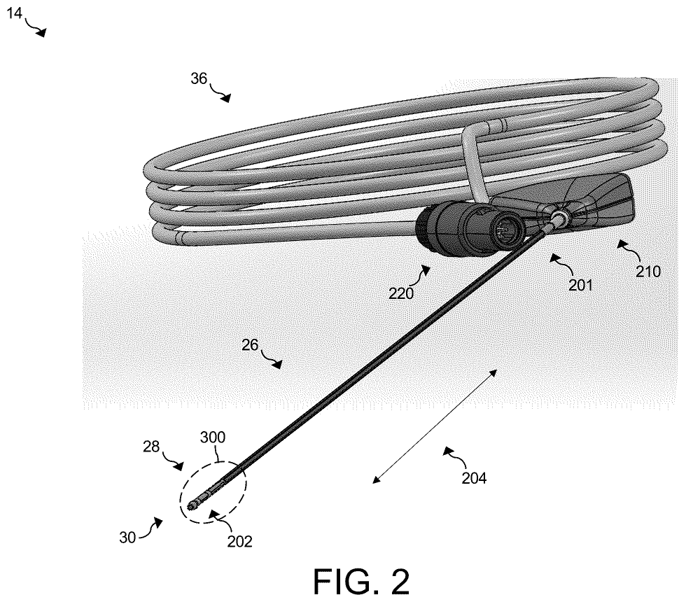

FIG. 2 is an isometric view of the catheter 14 according to an embodiment. The shaft 26 has a proximal end 201 and a distal end 202. The shaft 26 can extend from the proximal end 201 to the distal end 202 parallel to an axis 204. The shaft 26 can be flexible and/or bendable such that the shaft 26 includes one or more curves and/or bends.

A housing 210 is disposed and/or attached to the proximal end 201 of the shaft 26. The housing 210 can also be referred to as a connecting hub or a handle. The housing 210 can enclose electrical connections between the acoustic sensor(s) 28 and the cable 36. A proximal end of the cable 36 can include an electric plug 220 that can be electrically connected to a detector 42. The housing 210 can also include one or more ports to connect with respective channel(s) in the shaft 26.

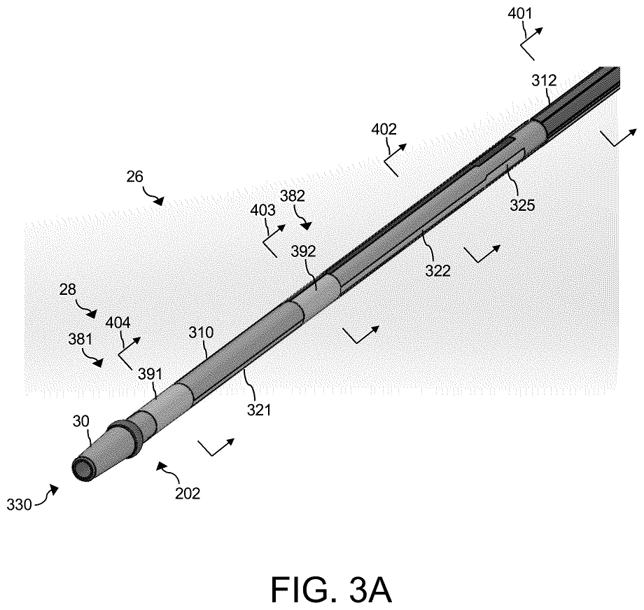

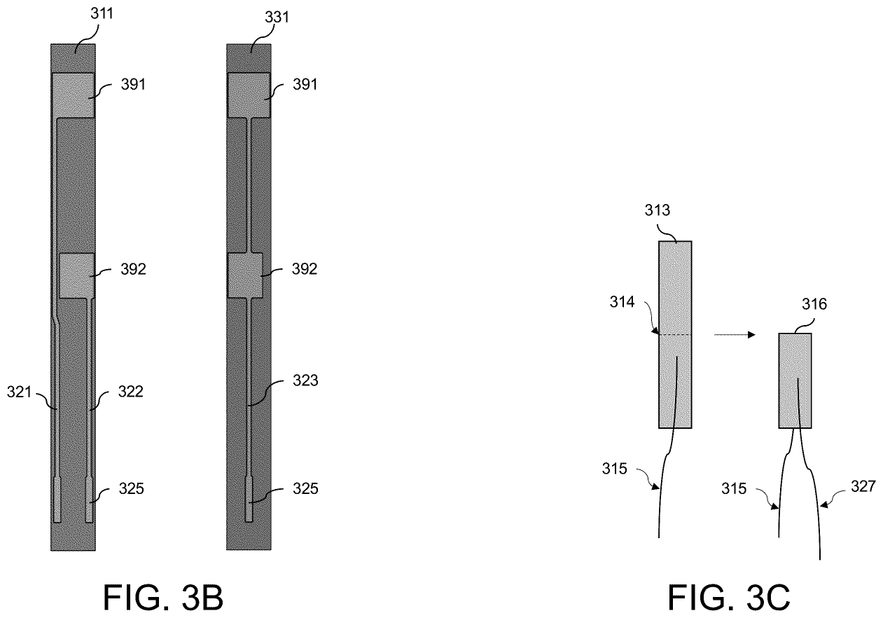

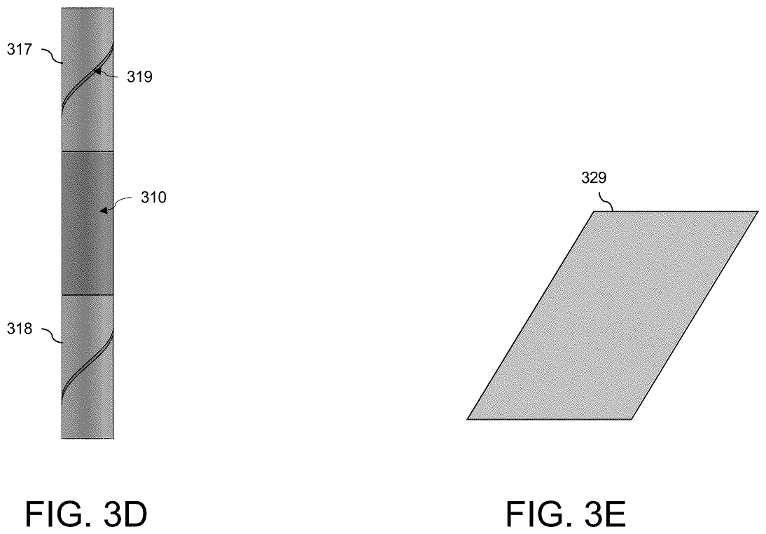

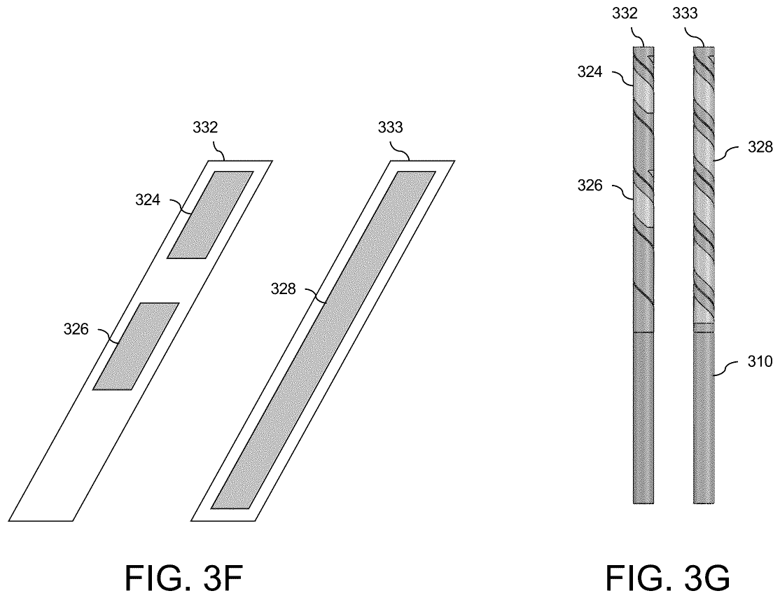

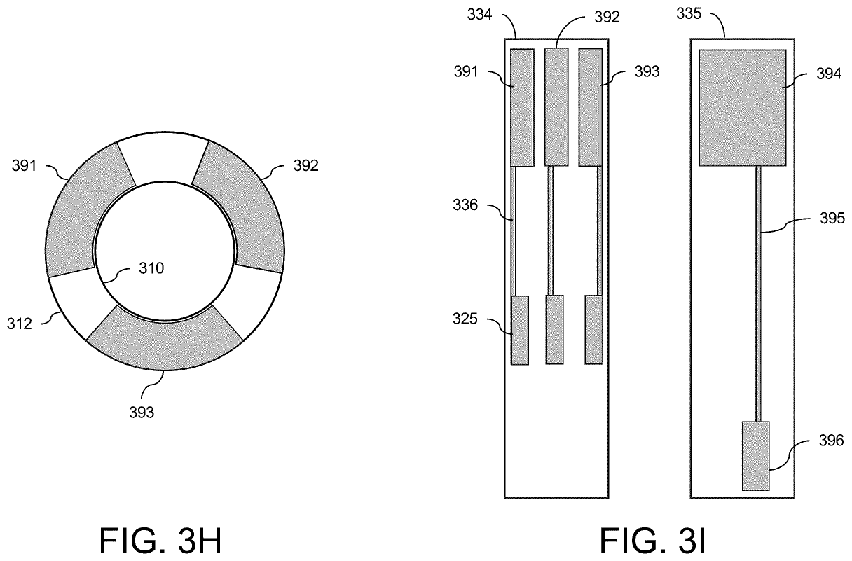

FIG. 3A is an enlarged view of region 300 in FIG. 2 which corresponds to the distal end of the catheter 14. In this embodiment, the acoustic sensor(s) 28 include a first acoustic sensor 381 and a second acoustic sensor 382. In other embodiments, the acoustic sensor(s) only include a first acoustic sensor 381 or a second acoustic sensor 382. In other embodiments, the acoustic sensor(s) 28 include more than two acoustic sensors.

The first acoustic sensor 381 includes a first piezoelectric polymer film 391 wrapped around and/or disposed on an inner tube 310 of the shaft 26. The second acoustic sensor 382 includes a second piezoelectric polymer film 392 wrapped around and/or disposed on the inner tube 310 of the shaft 26. The shaft 26 includes an outer tube 312 that is disposed over the inner tube 310. For illustration purposes only, the outer tube 312 is illustrated as not extending to the distal end 202 of the shaft 26 to not obscure the first and second sensors 381, 382. However, the outer tube 312 may extend to the distal end 202 of the shaft 26 (e.g., to a proximal end of the tip 30). The outer tube 312 can be configured to cover the first and second sensors 381, 382, including the first and second piezoelectric polymer films 391, 392, such that the first and second sensors 381, 382 (and the first and second piezoelectric polymer films 391, 392) are between the inner tube 310 and the outer tube 312. The inner tube 310 and the outer tube 312 can be arranged coaxially. In certain embodiments in which the inner tube comprises multiple lumens, the respective lumen axes may be generally parallel to the axis of the outer tube 312 but are not required to be strictly coaxial though they may be coaxial in one or more embodiments.

Electrical leads 321, 322 may be disposed on the piezoelectric film and/or the inner and outer tubes 310 and 312. A distal end of each electrical lead 321, 322 is electrically connected to a respective one or both of the first and second sensors 381, 382 (e.g., to one or both of the first piezoelectric polymer film 391 and/or the second piezoelectric polymer film 392, respectively). A proximal end of each electrical lead 321, 322 is electrically connected to one or more electrical contact pads 325. In some embodiments, the proximal end of each electrical lead 321, 322 is electrically connected to a respective electrical contact pad 325. In some embodiments, there can be more than two electrical leads 321, 322, such as three or more electrical leads electrically connected to one or more electrical contact pads 325. In an embodiment, there are three electrical leads (e.g., including electrical leads 321, 322) and three electrical pads 325, where each lead is electrically connected to a respective electrical pad 325. Electrical wires can be electrically connected to the electrical pads 325 and an electrical connection point in the housing 210. The electrical wires may comprise single or coaxial cables, a flexible printed circuit, a conductive foil, or a conductive coating on the catheter elements (e.g., the inner and/or outer tubes), and may be disposed between the inner tube 310 and the outer tube 312.

The first and second piezoelectric polymer films 391, 392 comprise or consist of a piezoelectric polymer such as polyvinylidene fluoride (PVDF), Pb(Zr,Ti)O3 (PZT), AlNPoly(vinylidene fluoride-trifluoroethylene) (PVDF-TrFE) copolymers, BaTiO3/PVDF-TrFE, ZnO films, Zr₂P₂BrCl, M₂CO₂, and/or another piezoelectric polymer. A piezoelectric polymer, such as PVDF, is a polarized fluoropolymer that, in its polar form (e.g., β-phase), demonstrates strong and stable piezoelectric and pyroelectric activities. Ferroelectricity is the property of having a spontaneous electric polarization that can be reversed by the application of an external electric field. This property is typically observed in materials with a non-centrosymmetric crystal structure that allows dipoles to be formed and switched within the crystal.

Piezoelectric polymer films, such as those comprising or consisting of PVDF, can provide ultrasonic performance at a low cost with consistent unit-to-unit repeatability. PVDF films can provide broad frequency bandwidths with low Q-factors and low electrical impedance (e.g., 30-100 Ohms). In addition, PVDF films are lightweight and flexible to conform with the cylindrical surface of the catheter 14 (e.g., of the inner tube 310 and/or outer tube 312). PVDF films can also provide excellent acoustic matching with liquids and/or biological tissues.

PVDF films are mechanically robust and are also used in structural and coating applications without specific need for piezoelectric properties. In an embodiment, the shaft 26 (or a portion of the shaft 26) can be formed out of PVDF (or another piezoelectric polymer material) and the acoustic sensor(s) 28 can be formed by depositing electrodes on certain locations of the PVDF structure.

The enlarged view also illustrates that the tip 30 can be tapered, for example, to facilitate easy passage of the catheter 14 along or through an anatomical feature such as a vessel (e.g., a blood vessel) or a ureter. The tip 30 includes an opening 330 that is coupled to one or more channels defined in the inner tube 310.

The acoustic sensor assembly at the distal end of the catheter, as shown in FIG. 3A, may include various configurations of piezoelectric elements positioned on the catheter shaft to detect incident acoustic waves. FIGS. 3B-3I illustrate several embodiments of the piezoelectric film sensor, representing variations in the arrangement, geometry, and/or wrapping of the piezoelectric film to enhance acoustic sensitivity, achieve omnidirectional response, and/or improve mechanical and electrical robustness. The following description details each of these embodiments, highlighting their structural differences, electrical configurations, and the resulting effects on sensor performance.

Several examples of possible designs of piezoelectric film sensors (e.g., made of polarized PVDF) are illustrated in FIGS. 3B-3E. FIG. 3B shows both the front and back surfaces (311 and 331, with opposite polarization (e.g., positive and negative, or vice versa), of a single piezoelectric film with two sensitive areas 391 and 392. These sensitive areas 391 and 392 are formed by the overlap of the conductive regions constructed on the opposite surfaces 311 and 331. In this example, the two sensitive areas 391 and 392 are depicted rectangular. The piezoelectric film having the opposite surfaces 311 and 331 is also depicted rectangular and it is assumed to be flat. The flat configuration typically represents the condition of the film prior to wrapping it around the inner tube 310 (shown in FIG. 3A) or placing it between the inner and outer tubes 310 and 312 (FIG. 3A). Note that both surfaces 311 and 331 include conductive regions that define the active sensing regions where they overlap. These active regions 391 and 392 are referred to as the sensing areas, sensing elements, or sensors. On surface 311, the sensing elements 391 and 392 are electrically connected to conductive leads 321 and 322, which connect each sensing element to its respective wire-attachment area 325. On the opposite surface 331, the sensing areas 391 and 392 are connected together via lead 323, which also electrically connects these sensing areas to the common ground pad 325. All leads and pads are arranged to avoid overlap when the film is wrapped around the catheter shaft. In one embodiment, the film may be partially wrapped around the inner tube 310 (FIG. 3A), or the width of the leads 322 and 323, pads 325, and active areas 391 and 392 may be made slightly smaller than the catheter circumference to prevent electrical shorting.

This configuration, however, may result in nonuniform sensitivity, with reduced sensitivity corresponding to the unwrapped region. To overcome this limitation, FIGS. 3C-3E illustrate alternative designs in which the piezoelectric film with its sensitive elements can be wrapped completely around the catheter shaft, forming a continuous 360° cylindrical sensing band. The advantage of these configurations is that they provide a uniform acoustic response regardless of catheter rotation or orientation relative to the acoustic source. To achieve fully omnidirectional sensitivity, including sensitivity in the axial direction, the dimensions of the sensitive elements are small compared with the wavelength of the acoustic wave. A typical design guideline is to maintain a minimum element dimension of approximately one-tenth ( 1/10) of the acoustic wavelength. While this approach provides an omnidirectional response, it can result in reduced overall sensitivity, since sensitivity is proportional to the total area of the active element.

FIG. 3C illustrates a folded piezoelectric film configuration designed to prevent electrical shorting when the film is wrapped circumferentially around the catheter shaft. In this embodiment, the typically flat piezoelectric film 313 is folded along the dashed line 314 so that the signal electrode and connecting wire 315 are sandwiched between the two layers of the piezoelectric film. As a result, the ground electrode 316 appears on both external surfaces of the folded film, while the signal-electrode lead and/or connecting wire 315 remains enclosed internally. Because the external surfaces of the folded configuration are electrically connected, the ground connector 327 can be attached to either external surface of the folded piezoelectric film 316. The folded geometry provides improved electrical shielding, thereby enhancing signal-to-noise performance during acoustic detection. Another significant advantage of this folded construction is that the film can be wrapped fully around the catheter tube (e.g., tube 310 in FIG. 3A) without causing electrical shorting between overlapping conductive regions of the film. Because both layers of the folded piezoelectric film are exposed to essentially the same acoustic pressure, the piezoelectrically induced electric charges generated by each layer are additive. Two or more piezoelectric layers may be folded in this manner to increase the overall output signal of the acoustic sensor.

FIG. 3D illustrates another embodiment in which piezoelectric film sensors 317 and 318 are wrapped around the catheter shaft 310 in a spiral or helical configuration. To enable the spiral wrapping, a typically flat piezoelectric film may be cut into a parallelogram shape 329, as shown in FIG. 3E The parallelogram geometry 329 allows wrapping the film without overlap, leaving only a small gap 319 (FIG. 3D) to prevent electrical shorting, while providing essentially continuous axial coverage and uniform rotational sensing along the catheter surface. The spiral configuration increases the effective sensing area and improves coupling with incident acoustic waves arriving from various angles, enabling the construction of catheter sensors with omnidirectional sensitivity. Furthermore, the helical wrapping provides flexibility in tuning both axial and circumferential sensitivity by adjusting the pitch angle and the width of the piezoelectric film.

FIG. 3F illustrates an alternative embodiment in which a parallelogram-shaped piezoelectric film incorporates two active sensing elements 324 and 326 on a single substrate. The conductive regions—electrodes 324 and 326—are located on one surface 332 (e.g., with positive polarization) of the piezoelectric film, while the common ground electrode 328 is located on the opposite surface (e.g., with negative polarization). The conductive area of the ground electrode 328 can be large and may cover essentially the entire surface 333. The sensing areas are defined by the regions where the conductive areas 324 and 326 overlap with the ground electrode 328. These overlap areas define two spatially separated sensing regions, allowing the sensor to detect acoustic waves at distinct axial positions along the catheter shaft when this dual-element piezoelectric film is wrapped around the catheter shaft 310, as shown in FIG. 3G.

FIG. 3G shows two options for wrapping a parallelogram-shaped piezoelectric film with the dual sensor (e.g., as shown in FIG. 3F) around a cylindrical shaft 310. In a first option, the ground electrode 328 faces the inside of the wrap, so that the positive leads 324 and 326 are on the outer surfaces 332. In a second option, the ground electrode 328 faces outward, making the outer surface of the wrap appear as surface 333 with the ground electrode 328 visible. Enclosing the signal electrodes 324 and 326 within the wrap of the ground electrode 328 in the second option provides better electrical shielding from EMI compared to the first option. These two options illustrate possible configurations of the inner and outer surfaces of the wrapped film, both of which provide circumferential acoustic coverage with improved spatial resolution and enhanced sensitivity compared with a single-element configuration.

Another embodiment employs multiple sensing elements arranged circumferentially around the catheter to provide independent detection of acoustic waves at different angular positions. FIG. 3H shows an example with three active sensing areas—391, 392, and 393—distributed along the circumference of the catheter. In one embodiment, the sensors 391, 392, and 393 may be positioned between the inner shaft 310 and the outer shaft 312. In another embodiment, the circumferentially arranged sensors 391, 392, and 393 may be offset along the axis of the catheter shaft 204 (FIG. 2) and/or combined with axially offset sensors, as shown in FIG. 3A. Signals detected by the circumferentially distributed sensors 391, 392, and 393 may be used to provide information—such as the phase and amplitude of the incoming wave—to determine the angular orientation of the incoming wave relative to the catheter axis.

FIG. 3I illustrates one possible example of a flat piezoelectric film with three sensitive regions—391, 392, and 393—prior to being wrapped around the catheter shaft (e.g., between the inner tube 310 and outer tube 312), as shown in FIG. 3H. The three active sensing elements 391, 392, and 393 are connected via individual leads 336 to wire attachment pads 325 on one surface of the film 334 (e.g., with positive polarization), while the common ground electrode 394 is positioned on the opposite surface 335 (e.g., with negative polarization). On that side of the film, the common ground electrode 394 is connected via lead 395 to wire-attachment pad 396. By analyzing the relative amplitude and/or phase delay of the signals detected by these elements, the direction of an incoming acoustic wave can be determined. This configuration enables independent signal detection at each sensor location, providing full three-dimensional spatial resolution of the catheter position even when used with a linear array transducer. The circumferentially distributed sensor elements thus break the symmetry that would otherwise occur with linear transducers and axially aligned sensors, enabling more precise localization of acoustic sources.

In some embodiments, the sensing elements can be directly connected to electrical conductors, such as wires or cables, to transmit the electrical signal along the catheter shaft. In other embodiments, the sensing elements are electrically connected to conductive leads along the piezoelectric film, and the electrical wires are connected to these leads. This configuration allows for the separation of the sensor elements from the wire connections, providing greater flexibility in sensor placement and assembly. By decoupling the sensing elements from the wires, one can reduce mechanical stress on the sensors, improve manufacturability, and facilitate modular assembly or replacement of sensor components without disturbing the wiring along the catheter shaft.

The conductive leads on the piezoelectric film may be formed using various techniques, including vacuum deposition of conductive metals, application of conductive paint, or deposition of a conductive layer followed by patterning the leads using chemical etching or laser ablation. In one or more embodiments, vacuum deposition may be performed in multiple steps to optimize adhesion, conductivity, and durability. For example, a thin layer of chromium, which exhibits strong adhesion to PVDF, may first be deposited onto the film surface. A gold layer with a thickness of approximately 0.05 μm to 0.15 μm can then be deposited over the chromium layer to provide excellent electrical conductivity and resistance to corrosion.

In alternative embodiments, conductive paint, such as a silver or graphite paste, may be applied directly to the PVDF film using methods including screen printing, brushing, or spraying, followed by curing to form stable, conductive leads. The thickness of the conductive paint is typically on the order of 10 μm. In yet another approach, a continuous conductive layer may first be deposited over the PVDF film and subsequently patterned to define the desired lead geometry using chemical etching or laser ablation techniques. These methods allow precise control of electrode shapes and spacing while minimizing the risk of electrical shorting and maintaining the integrity of the piezoelectric material. The conductive paint may be applied either before or after positioning the piezoelectric film on the catheter shaft, depending on the desired fabrication sequence and sensor configuration. Alternatively, or additionally, undesired portions of the conductive paint may be removed from the piezoelectric film using suitable solvents, such as acetone.

In additional embodiments, the conductive leads may be formed using a conductive foil. For example, a copper foil, for example 10 μm to 30 μm in thickness, with a conductive adhesive layer can be applied either before or after wrapping the piezoelectric film around the catheter shaft. In some configurations, the conductive foil may serve as one of the electrodes, such as a common ground, while the opposing electrode is formed using printed or sputtered conductive material.

In some embodiments, some or all of the conductive leads, conductive pads, and sensitive areas may be formed by conductive regions on the inner and/or outer catheter shafts, which can provide corresponding leads, pads, and/or sensitive elements through capacitive coupling. These conductive regions can be made by printing conductive material and/or other methods described above, such as using a conductive foil. An additional advantage is that the outer member of the catheter, when covered with a conductive material, provides electrical shielding against electromagnetic interference. Alternatively, the electrical wires can be 42 AWG coaxial cables (approximately 0.064 mm in diameter), providing shielding from electromagnetic interference (EMI) and adequate signal transmission while maintaining flexibility and minimizing mechanical stress on the sensor assembly.

Electrical wires may be connected to the conductive foil or conductive leads on the PVDF film using various techniques, including application of conductive paint, conductive epoxy, attachment with conductive adhesive, or soldering. Soldering can be performed using a low-temperature solder or solder paste to avoid damaging the piezoelectric film. Examples of suitable low-temperature solders include alloys containing silver for improved mechanical and electrical properties, such as Sn 42/Bi57/Ag1, which has a melting point of approximately 138° C. (280° F.). This melting temperature is below the melting point of PVDF, which is approximately 177° C., and also below its Curie point. However, depolarization of PVDF begins at temperatures around 72° C., and piezoelectric properties may start to decrease due to relaxation of polarization in the amorphous phase. To avoid or minimize depolarization, heat should be applied only to the portion of the connection comprising the conductive leads and electrical wires, rather than to the piezoelectric film itself.

The use of solder paste is particularly advantageous, as the paste typically consists of small, uniformly sized solder beads (e.g., 15-25 μm) suspended in flux. A thin layer of solder paste can be applied and melted using a temperature-controlled heat gun or an integrated hot/cold system. These integrated systems, also known in industry as “hotboxes,” are routinely used in catheter manufacturing and specifically designed for precise thermal processing. They incorporate a heating phase, using hot air or a thermal nozzle, and a controlled cooling phase to rapidly set the material after shaping, bonding, or soldering. Using a hotbox to melt and cool the solder paste allows precise control of the connection thickness between the leads and the electrical wires. In addition, various masks or stencils can be used to ensure a predetermined pattern and to prevent overheating of sensitive elements.

Alternatively, the soldering can be performed using electrically conductive heating. In this approach, electrical coils generate eddy currents in the conductive elements, which heat the metal leads and pads directly without significantly heating the piezoelectric film. This method allows precise and localized heating of the conductive regions, reducing the risk of damaging the temperature-sensitive piezoelectric material while ensuring reliable solder connections. In contrast, conventional soldering methods (such as using a hot-air gun or heat block) heat both the solder and the surrounding materials, including the piezoelectric film, which can increase the risk of depolarization or mechanical damage. By selectively heating only the conductive pads located at a distance from sensitive elements, electrically induced heating provides greater process control, reproducibility, and protection of the piezoelectric film, making it particularly advantageous for sensor assemblies.

Collectively, the PVDF film sensor embodiments described herein provide enhanced acoustic detection capabilities at the distal end of the catheter. By varying the arrangement, geometry, and wrapping of the film, these designs achieve desirable sensitivity while minimizing electrical shorting and improving mechanical robustness. The folded and spiral configurations further increase the effective sensing area and provide additive piezoelectric response, resulting in enhanced signal output and improved signal-to-noise performance. The multi-element and circumferentially distributed sensor designs also enable spatial localization of acoustic signals. These advantages enable more reliable and precise detection of incident acoustic waves, thereby enhancing the overall performance and efficacy of the catheter-based acoustic sensing system.

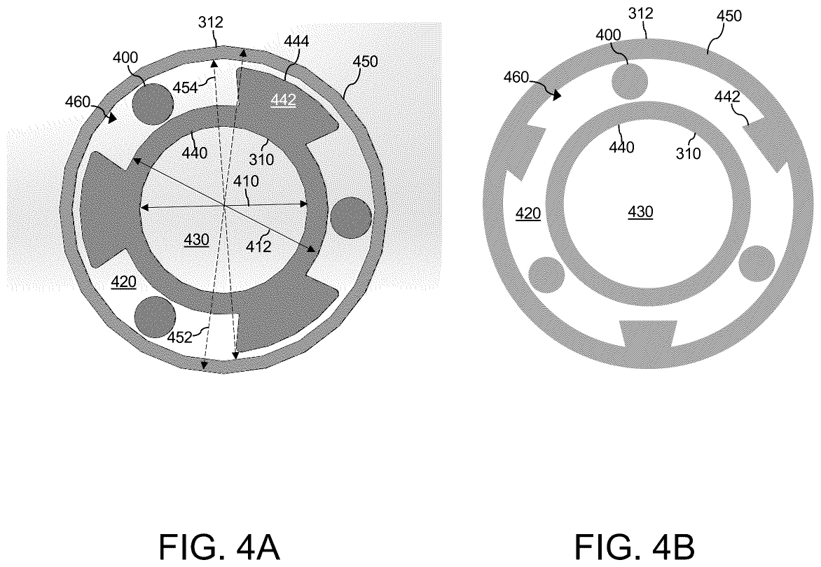

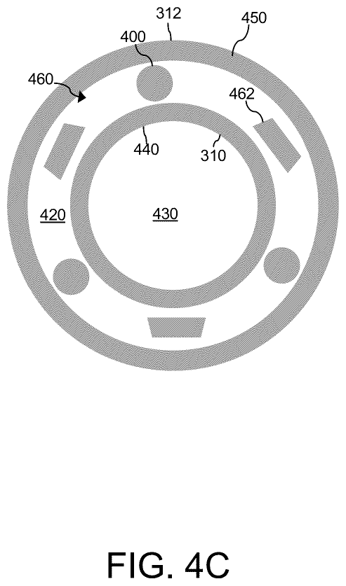

FIG. 4A is a cross section of the shaft 26 by plane 401 in FIG. 3. The cross section is the shaft 26 between the electrical pads 325 and the proximal end 201 of the shaft 26. The cross section illustrates that one or more (e.g., a plurality of) wires 400 or other electrical conductors are disposed between the inner tube 310 and the outer tube 312. In the illustrated embodiment, there are three wires 400. In other embodiments, there can be only one wire 400, two wires 400, four wires 400, or another number of wires. The wires 400 can be electrically connected to respective electrical pads 325. In some embodiments, a wire 400 can be electrically connected to more than one electrical pad 325 and/or multiple wires 400 can be electrically connected to the same electrical pad 325.

Wires 400 can be either electrically insulated or exposed wires. In addition, an insulator material 420 can be disposed between the inner tube 310 and the outer tube 312 and can surround each wire 400 to provide electrical insulation thereto. The insulator material 420 can also improve the mechanical strength of the shaft 26.

One or more channels 430 is/are defined by at least an inner diameter 410 of the inner tube 310. The channel(s) 430 can be used to introduce a guidewire, a tool, an acoustic enhancer (e.g., engineered microbubbles), fluids, and/or a therapeutic substance to or near a target volume. The channel(s) 430 extend to the opening 330 at the tip 30.

The inner tube 310 has a wall 440 having an inner-tube thickness that can be defined by the difference between an outer diameter 412 and the inner diameter 410 of the inner tube 310. The radial thickness of the wall 440 can have one or more localized regions 442 of increased thickness compared to the inner-tube thickness. For example, localized segments 444 of the wall 440 can have a larger localized outer diameter or outer radius compared to the outer diameter 412 (or outer radius). The localized regions 442 can be regions (e.g., extrusion regions) where additional material forming the wall 400 is extruded during manufacturing. The localized regions 442 increase the structural strength and/or rigidity of the inner tube 310 and can increase the structural strength and/or rigidity of the shaft 26 as a whole. Additionally or alternatively, the localized regions 442 can be disposed between neighboring wires 400 to physically separate and electrically isolate the wires 400.

The outer tube 312 has a wall 450 having an outer-tube thickness that can be defined by the difference between an outer diameter 452 and an inner diameter 454 of the outer tube 312. A gap 460 is defined between the outer diameter 412 of the inner tube 310 and the inner diameter 454 of the outer tube 312. The wire(s) 400, the insulator material 420, and the localized regions 442 are disposed in the gap 460.

In other embodiments, the localized regions 442 can be on the wall 450 of the outer tube 312 instead of on the wall 440 of the inner tube 310, for example as illustrated in FIG. 4B, which is a cross section of the shaft 26 by plane 401 in FIG. 3 according to another embodiment. The localized regions 442 increase the structural strength and/or rigidity of the outer tube 312. The cross section illustrated in FIG. 4B is the same as the cross section illustrated in FIG. 4A except for the location of the localized regions 442, and thus not all features of the cross section illustrated in FIG. 4B are labelled as they are in FIG. 4A. In other embodiments, the localized regions 442 can be on both the wall 450 of the outer tube 312 and the wall 440 of the inner tube 310.

In other embodiments, the localized regions 442 can be replaced by spacers 462 that are constructed as an independent element and are not necessary a constituent part of the wall 450 of the outer tube 312 and/or of the wall 440 of the inner tube 310, as illustrated in FIG. 4C. The number, geometry, and position of the localized regions 442 or spacers 462 can vary, as would be apparent for those skilled in the art. Alternatively, the localized regions 442 or spacers 462, or both can be removed.

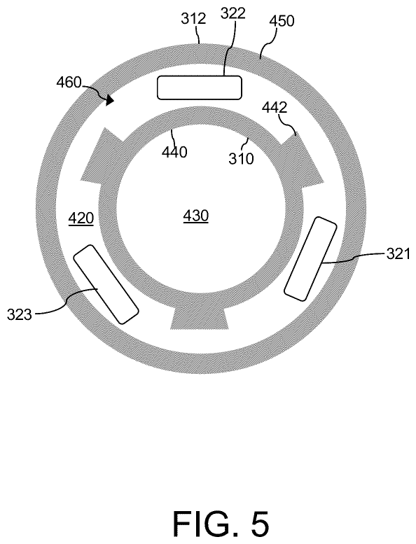

FIG. 5 is a cross section of the shaft 26 by plane 402 in FIG. 3. The cross section illustrated in FIG. 5 shows three electrical leads 321-323 disposed in the gap 460 between the wall 450 of the outer tube 312 and the wall 440 of the inner tube 310. There can be additional or fewer electrical leads 321-323 in other embodiments. The localized regions 442 are shown as being disposed on the wall 440 but can be on the wall 450 instead of or in addition to the wall 440, as discussed above. Alternatively, the localized regions 442 can be replaced with spacers 462 or can be removed.

The cross section illustrated in FIG. 5 is the same as the cross section illustrated in FIG. 4A except that the wires 400 are replaced with electrical leads 321-323, and thus not all features of the cross section illustrated in FIG. 5 are labelled as they are in FIG. 4A.

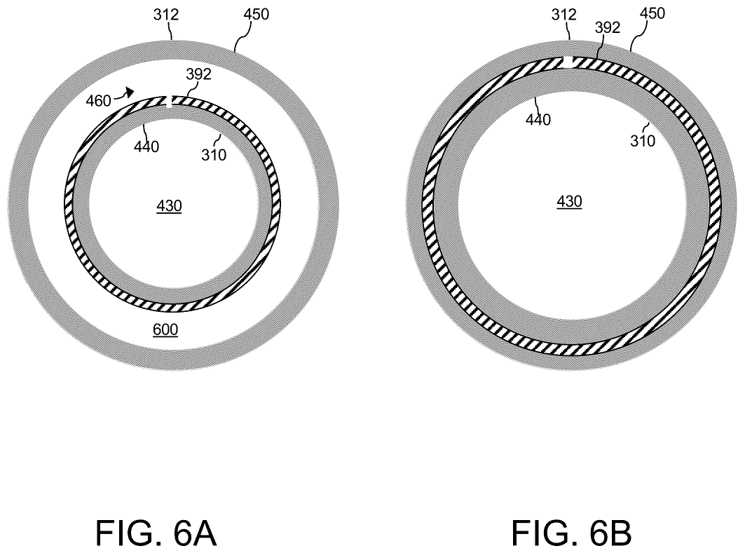

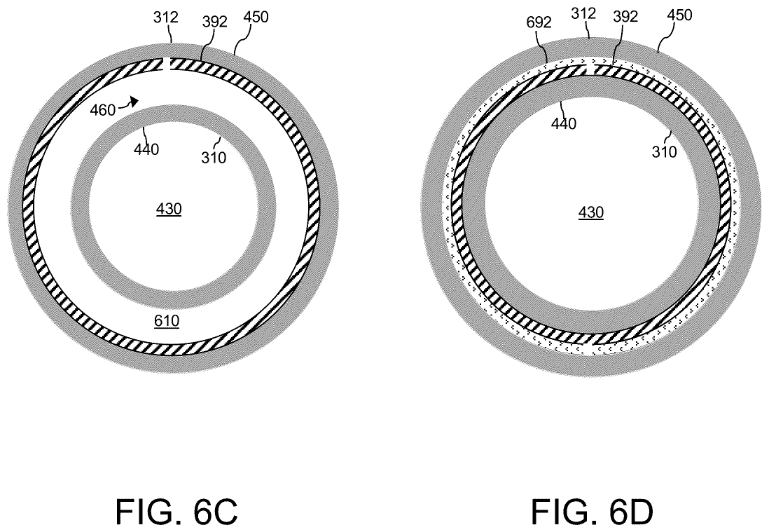

FIG. 6A is a cross section of the shaft 26 by plane 403 in FIG. 3. The cross section illustrated in FIG. 6A shows the second piezoelectric polymer film 392 disposed in the gap 460. The second piezoelectric polymer film 392 can be disposed about and/or cover the circumference of the wall 440 of the inner tube 310. In other embodiments, the second piezoelectric polymer film 392 can be disposed about and/or cover a portion (e.g., an arc) of the circumference of the wall 440, such in the range of about 30% to about 75%, including any values or ranges therebetween, of the circumference of the wall 440. The localized regions 442 and spacers 462 do not extend to this portion of the shaft 26 to allow the second piezoelectric polymer film 392 to be disposed about and/or cover the inner tube 310 without forming air gaps and/or to minimize air gaps. Good acoustic transmission requires an absence (or minimization) of air gaps between the outer tube 312 and the sensor (e.g., second piezoelectric polymer film 392). This can be achieved, for example, by filling the gap 460 with one or more materials 600 that have an acoustic impedance that matches (e.g., within about 20%) the impedance of bodily fluids and/or tissue, which have an acoustic impedance similar to that of water. In another embodiment, air gaps can be removed or reduced by the locally increased the diameter of the inner tube 310 so that the second piezoelectric polymer film 392 is in direct physical contact with the outer tube 312 as well as with the inner tube 310, as illustrated in FIG. 6B. In another embodiment, air gaps can be removed or reduced by disposing the second piezoelectric polymer film 392 about and/or cover the inner diameter of the outer tube 312, as illustrated in FIG. 6C. The gap 460 between the second piezoelectric polymer film 392 and the inner tube 310 can be filled with one or more materials 610 having a high acoustic impedance and/or that can provide an acoustically rigid interface to increase acoustic pressure at the acoustic sensor. In some embodiments, the material(s) 610 can be configured to absorb acoustic waves. Partial or complete absorption of acoustic waves (such as by an epoxy resin) can reduce or eliminate the reflection of acoustic waves, which may be desirable in some embodiments.

In some embodiments, two piezoelectric polymer films 392, 692 can be stacked and disposed about the wall 440 of the inner tube 310, for example as illustrated in FIG. 6D. One of the two piezoelectric polymer films can be reversed biased to reduce electromagnetic interference (EMI) and increase signal-to-noise ratio (SNR). The piezoelectric polymer films 392, 692 can be the same as or different than each other. To remove or reduce air gaps, the outer piezoelectric polymer film 692 can be in direct physical contact with the outer tube 312, as illustrated in FIG. 6B, or any gap between the outer piezoelectric polymer film 692 and the outer tube 312 can be filled with one or more materials 600 in the same manner as illustrated in FIG. 6A. Additionally or alternatively, any gap between the inner piezoelectric polymer film 392 and the inner tube 310 can be filled with one more materials 610 as illustrated in FIG. 6C. The inner and outer piezoelectric polymer films 392, 692 can be in direct physical contact with each other. The inner piezoelectric polymer film 392 can be in direct physical contact with the inner tube 310.

The localized regions 442 are shown as being disposed on the wall 440 but can be on the wall 450 instead of or in addition to the wall 440, as discussed above. Alternatively, the localized regions 442 can be replaced with spacers 462 or can be removed.

The cross sections illustrated in FIG. 6A-D are the same as the cross section illustrated in FIGS. 4A, 4B, or 4C except that the wires 400 are replaced with the piezoelectric polymer film 392 and the optional piezoelectric polymer film 692, and the insulator material 420 is replaced with acoustically transparent material(s) 600 or the gap 460 is removed by increasing the diameter of the inner tube 310, and thus not all features of the cross sections illustrated in FIG. 6A-D are labelled as they are in FIG. 4A-C.

A cross section through plane 404 in FIG. 3 can be the same as any of the cross sections illustrated in FIG. 6A-D except that in the cross section through plane 404 the second piezoelectric polymer film 392 is replaced with the first piezoelectric polymer film 391.

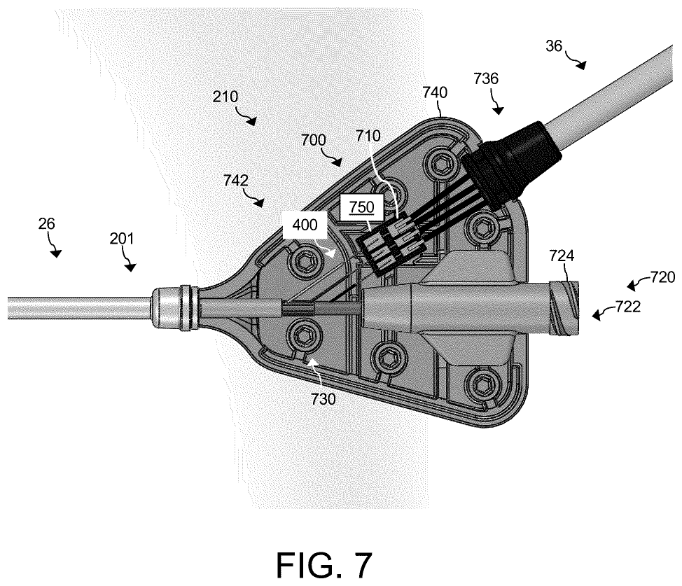

FIG. 7 is a top view of the housing 210 with a top cover removed to expose the inside of the housing 210. Electronics 700 are disposed in the housing 210. The electronics 700 can include devices with respective housings and/or electrical circuits mounted on a printed circuit board (PCB) 710. For example, the wires 400 that are electrically connected to the electrical contact pads 325 (FIG. 3) can be electrically connected to the electronics 700 (e.g., to the PCB 710). The electronics 700 can include electrical circuits to precondition the signal, improve the signal-to-noise ratio of the signals, and/or reject common-mode electromagnetic interference. In some embodiments, the electrical circuits can include one or more signal preamplifiers, common-mode chokes, and/or or common-mode rejection transformers. The wire connection (e.g., of the wires 400 to the electronics 700) in the housing 210 can be either direct to provide continuity for direct current (DC) or indirect with capacitive and/or inductive coupling to transmit only alternative current (AC). In addition, the electrical circuits can be used to electrically isolate the acoustic sensor(s) 28 from the detection circuitry (e.g., in an external detector such as detector 42 (FIG. 1)) by using an isolating device, such as, for example, isolation transformers or optical couplers.

An optional cable 36 can be electrically connected to the PCB 710 to receive the electrical signals after passing through the electronics 700. Thus, the housing 210 can provide a physical and electrical transition from the delicate wires 400 connecting the acoustic sensor(s) 28 to a more robust electric cable 36. The cable 36 can be connected to the housing using either another connector or permanently for example using a strain-relief device 736. The cable 36 may include four conductors corresponding to the two independent electrical connections required for each of the two sensors (i.e., two conductors per sensor, for a total of four). The conductors may be implemented using any combination of standard insulated wires, twisted pairs, and/or coaxial cables. In certain embodiments, twisted pairs and coaxial cables provide improved electromagnetic interference (EMI) immunity and reduced sensor-to-sensor crosstalk.

The cable 36 can incorporate all conductors within a conductive shield—typically one or more coaxial shielding layers—to further reduce EMI and minimize coupling between the sensor channels. FIG. 7 illustrates an embodiment in which four wires emerge from the cable 36, with the two ground wires for the respective sensors connected to a common conductive pad on the PCB 710. This conductive pad is then connected to a single ground conductor (one of the wires 400) extending along the catheter shaft 26. In this configuration, only three conductors are required along the shaft 26. Although combining the grounds in this manner allows the use of three conductors along the catheter shaft, in other embodiments each sensor can be connected using a dedicated coaxial cable to further reduce EMI, enhance signal integrity, and minimize crosstalk between the sensors. In these other embodiments, the PCB 710 may include either three pads when a combined common ground is used, or four separate pads (not shown) when twisted pairs and/or coaxial cables are kept fully insulated from one another.

Additionally or alternatively, the electrical signal data, representing the acoustic signals received by the acoustic sensor(s) 28, can be transmitted wirelessly using wireless communication circuitry 750 that can be mounted on and/or electrically coupled to the PCB 710. The wireless transmission circuitry 750 can be configured to transmit using a local wireless protocol such as Bluetooth, a local wireless network such as WiFi, a wide area wireless network such as a cellular network, or another wireless transmission network or protocol.

The housing 210 can include a port 720 that has an opening 722 connected to the channel(s) 330 in the shaft 26. The port(s) 720 can be configured to connect to a tube or syringe for example using a Luer-lock fitting 724. The Luer-lock fitting 724 is shown as a female fitting but can be a male fitting in another embodiment. The Luer-lock fitting 724 can provide a leakproof connection with a standard male-taper fitting of a syringe tips and/or other fluid-transfer devices.

The opening 722 can be used to insert a guidewire, a tool, an acoustic enhancer (e.g., engineered microbubbles), fluids, and/or a therapeutic substance through the channel(s) 430 and out the opening 330 in the tip 30 (FIG. 3), for example to be placed at or near a target volume 40.

The housing 210 can include pins 730 to releasably attach the front (not illustrated) and the back 740 of the housing 210. The internal space 742 of the housing 210 can be potted to provide a fluid seal (e.g., for liquids and/or gasses), as well as to improve mechanical and electrical robustness of the final assembly.

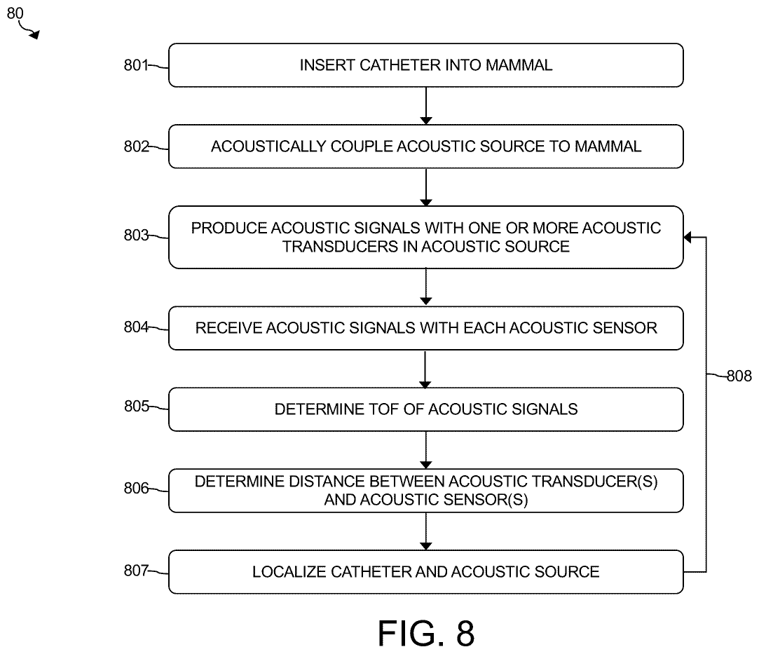

FIG. 8 is a flow chart of a method 80 for localizing an acoustic source 12 and a catheter 14 with respect to each other.

In step 801, the catheter 14 is inserted into a natural or surgical opening 32 in a mammal.

In step 802, the acoustic source 12 is acoustically coupled to a mammal 22 such as a human. The acoustic source 12 can be placed directly on the mammal 22 (e.g., on the skin 20 of the mammal 22) to acoustically couple the acoustic source 12 to the mammal 22. Alternatively, an acoustically transmitting media 24, such as water, a water cushion, an acoustic coupling oil, and/or an acoustic coupling gel, can be disposed between (e.g., in direct physical contact with) the acoustic source 12 and the skin 20 to improve acoustic transmission and acoustic coupling. The acoustic source 12 can be acoustically coupled at a position on the mammal 22 that corresponds to a target location of the introduced catheter 14.

In step 803, acoustic signals 34 are produced with one or more acoustic transducers 18 in the acoustic source 12. When the acoustic signals 34 are produced with multiple (e.g., two or more) acoustic transducers 18, the acoustic signals 34 are sequentially produced with each acoustic transducer 18.

The acoustic source 12 can include an array or another configuration of acoustic transducers 18. The array can include one row (e.g., a linear array) or two or more rows of acoustic transducers 18. In some embodiments, one or more of the acoustic transducers 18 can be positionally offset with respect to a linear array to break the symmetry of the acoustic transducers 18.

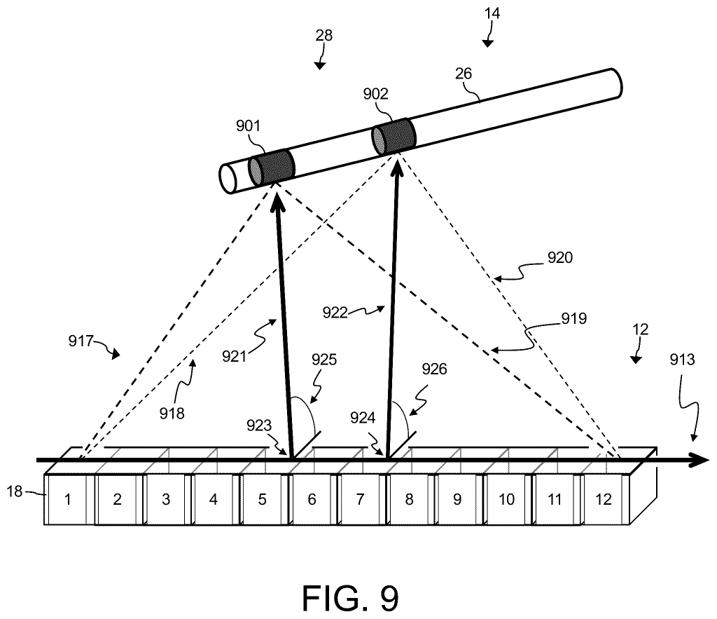

FIG. 9 illustrates an example linear array of acoustic transducers 18 in an acoustic source 12 and an example catheter 14, which is simplified as a cylinder for illustration purposes only. The acoustic signals 34 are sequentially produced by the first and last (numbers 1 and 12) acoustic elements. In other embodiments, the acoustic signals 34 can be sequentially produced by other and/or additional acoustic transducers 18. In other embodiments, the acoustic signals 34 are only produced by one acoustic transducer 18.

In step 804, the acoustic signals 34 are received by the acoustic sensor(s) 28 on or in the catheter 14. For example, in FIG. 9 the acoustic signals 34 are received by a first acoustic sensor 901 and a second acoustic sensor 902. When the acoustic signals 34 are sequentially produced by multiple acoustic transducers 18, the acoustic signals 34 are sequentially received by the first and second acoustic sensors 901, 902 from each acoustic transducer 18.

In step 805, the ToF(s) of the acoustic signals 34 transmitted between the acoustic transducer(s) 18 and the acoustic sensor(s) 28 is/are determined. The ToF(s) can be determined by a detector 42, such as a computer, that is electrically coupled to, in electrical communication, and/or in electromagnetic (e.g., wireless) communication with the acoustic sensor(s) 28. The detector 42 can be electrically coupled to, in electrical communication, and/or in electromagnetic (e.g., wireless) communication with a controller 25 and/or the source 12. The information from the detector 42 can be transmitted to a processing device and/or a display device.

The ToF can be determined as the arrival time of acoustic signal 34 to the acoustic sensor(s) 28 relative to a trigger signal provided by the controller 25. Alternatively, the ToF can be determined by measuring the time delay between the emitted and received signals. Alternatively, the ToF can be determined from the receiver signal alone because the acoustic sensor(s) 28 typically detects both the acoustic signal and EMI induced during the emission of the acoustic signals 34 by the emitting acoustic transducer(s) 18, such as a treatment head. The delay between the EMI and the detected acoustic signal at the acoustic sensor(s) 28 can be used to determine the ToF.

The delay between the emitted and received signals can be measured by finding the maximum of cross-correlation function of the emitted and received signals. Alternatively, the delay can be determined as a time difference between some characteristic features detectable in the emitted and received signals. The characteristic features can include a maximum amplitude or a certain threshold level, for example, in a range of about 10% to about 50% of the maximum amplitude. The characteristic features can also include signal peaks, valleys, and/or zero-crossings. These features can be used alone or in combination using sequences at one or plurality of frequencies. Furthermore, characteristic features can be created in acoustic waves using, for example, amplitude, phase, and/or frequency modulation. Some combination of these techniques may be used to improve the accuracy of the measurement of the delay time.

In yet another embodiment, the location of the sensor can be determined by considering the difference in arrival time from different transducer elements. The difference lies on a hyperbola with foci at the transducer locations. Different pairs of transducers can provide different hyperbolas. The intersection of the hyperbolas gives the location of the sensor. The mathematical formulation would be similar to that used in hyperbolic navigation.

In step 806, the distance between each acoustic sensor(s) 28 and each acoustic transducer(s) 18 is/are determined. The distance can be determined by multiplying the respective ToF by the speed of sound in soft tissue, which can be approximated as the speed of sound in water. The speed of sound in water at normal body temperature (37° C. or 98.6° F.), is 1524 m/s, about 2.6% greater than that at room temperature of 20° C.

In step 807, the catheter 14 and acoustic source 12 are localized with respect to each other. The localization resolution can vary depending on the number and/or configuration of acoustic transducers 18, including the number of transducer elements of the acoustic transducer(s) 18, that produce the acoustic signals 34 and/or the number of acoustic sensors 28. It is noted that the localization occurs without imaging, including acoustic (e.g., ultrasound) imaging. The relative positions of the acoustic source 12 and the catheter 14 can be displayed on a display screen 44 (FIG. 1) either wirelessly connected or in electrical communication with the detector 42.

In some embodiments, steps 803-807 can be repeated 808 such as during a medical procedure. Additionally or alternatively, the catheter 14 can be used to monitor the magnitude and/or other properties of therapeutic ultrasound (or other acoustic energy) produced by the acoustic source 12. The catheter 14 can also be used to introduce a guidewire, a tool, an acoustic enhancer (e.g., engineered microbubbles), fluids, and/or a therapeutic substance. Additionally or alternatively, steps 803-807 can be repeated 808 until the localization (e.g., roll, slide, and depth) are at a target value or within a target range. After the catheter 14 and acoustic source 12 are localized at a target value or within a target range, the position of the acoustic source 12 can be fixed or locked for example using a positioning accessory.

An example localization method is illustrated in FIG. 9. A reference axis 913 is positioned along the surface of the example linear array of acoustic transducers 18 of the acoustic source 12. Lines 921, 922 represent the radial distances, along respective radial lines, from the reference axis 913 to the first and second acoustic sensors 901, 902 on the catheter 14. Lines 921, 922 are orthogonal to the reference axis 913 and are oriented at respective angles 925 and 926 which can be the same or different from each other depending on the orientation of the catheter 14. Angles 925, 926 represent the angular coordinates of the first and second acoustic sensors 901, 902 in a cylindrical coordinate system with the longitudinal axis 913.

Measurements of ToF of the acoustic signals 34 from the acoustic transducers 18 to the first and second acoustic sensors 901, 902 allows one to determine the distance between the first and second acoustic sensors 901, 902 and acoustic transducers 18 (e.g., reference axis 913). Any pair of acoustic transducers 18 can be used to determine the following two coordinates of the first and second acoustic sensors 901, 902: the axial coordinates 923, 924 along the reference axis 913 and the radial distances 921, 922, respectively, from the reference axis 913. For example, distances from acoustic elements 1 and 12 of transducer 18 to the first and second acoustic sensors 901, 902 are shown with dashed lines 917-920. Considering the triangle formed by lines 917, 919, and a line (e.g., a segment of line 913) connecting the acoustic elements 1 and 12 of transducer 18, one can find the radial distance 921 between the first acoustic sensor 901 and the reference axis 913. Likewise, considering the triangle formed by lines 918, 920, and the line connecting the acoustic elements 1 and 12 of transducer 18, one can find the radial distance 922 between the second acoustic sensor 902 and the reference axis 913.

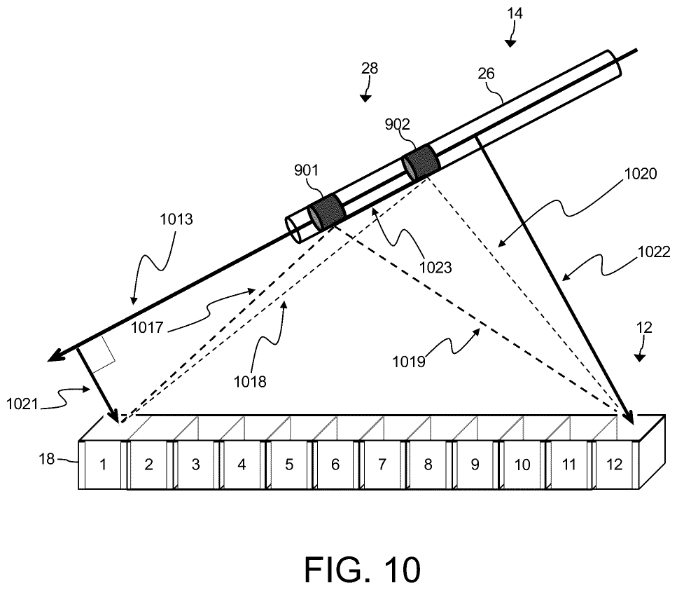

FIG. 10 illustrates an example localization method according to another embodiment. FIG. 10 represents a system of cylindrical coordinates with a reference axis 1013 passing along or through the catheter 14, where the catheter 14 remains still while the acoustic transducer 18 is movable. Lines 1021, 1022 represent the radial distances, along respective radial lines, from the reference axis 1013 to acoustic elements 1 and 12 of transducer 18, respectively. The distances from acoustic elements 1 and 12 of transducer 18 to the first and second acoustic sensors 901, 902 are shown with dashed lines 1017-1020. The radial distance 1021 from the reference axis 1013 to acoustic element 1 can be determined considering the triangle formed by lines 1017, 1018, and a line 1023 (e.g., a segment of reference axis 1013) that connects the first and second acoustic sensors 901, 902. Likewise, the radial distance 1022 from the reference axis 1013 to acoustic element 12 can be determined considering the triangle formed by lines 1019, 1020, and 1023. Lines 1021 and 1022 are generally skewed lines. That is, the triangle formed by lines 1017, 1018, and 1023 is not necessarily in the same plane with the triangle formed by lines 1019, 1020, and 1023.

One potential issue with the example localization methods illustrated in FIGS. 9 and 10 is that the symmetry of the linear array of acoustic transducers 18 may make it difficult to resolve all three coordinates of the respective position of each acoustic sensor 901, 902. All elements of a linear-array transducer are arranged along one line. The symmetry of the linear-array arrangement can pose a problem for a full three-dimensional position resolution. This problem is illustrated, for example with reference to in FIG. 9. Measurements of the distances between each acoustic sensors 901, 902 and any pair of acoustic transducers 18 allow determination of only two independent coordinates. In a cylindrical coordinate system, these coordinates are the position along the reference (or longitudinal) axis 913 and the radial distance (e.g., along lines 921, 922) from the reference axis 913. For the first acoustic sensor 901, for example, the radial distance from the reference axis 913 is shown with 921. The dashed lines 917, 919 connect the first acoustic sensor 901 with the acoustic elements 1 and 12, respectively, and represent the respective distance between the first acoustic sensor 901 and the acoustic elements 1 and 12. Consideration of any other colinear elements (for example, any of acoustic elements 2-11) does not resolve the angular coordinate 925—the third independent coordinate of the cylindrical system—because all the transducer elements are on the same line and in the same plane as lines 917 and 919. To determine the angle 925, one needs additional information.

In an embodiment, the problem associated with the symmetry of a linear-array arrangement can be resolved using either a 2D or 3D arrangement of the acoustic sensors 28 (e.g., acoustic sensors 901, 902) and/or of the acoustic transducers 18. For example, the catheter 14 can have three or more noncolinear acoustic sensors 28. Alternatively, the acoustic transducers 18 can be arranged either on a 2D surface or a 3D geometry. Note that to resolve the symmetry problem, it is sufficient to break the symmetry of a linear array by either separating, splitting, or adding one or more elements in the direction normal to the longitudinal axis (reference axis 913 in FIG. 9) of the linear array. For example, the acoustic transducers 18 can be arranged as a two-dimensional array or a three-dimensional array. Alternatively, one or more additional acoustic transducers 18 can be added to in a noncolinear manner to the linear array.

Another approach for localization is to employ a known distribution of acoustic field produced by a given transducer. Matching the known acoustic distribution with the measurements of the acoustic signal by the catheter sensors (e.g., acoustic sensors 28) allows one to determine the position of the catheter in the acoustic field. One example of this general approach is to use a known angular dependence of acoustic signal to assess the third independent coordinate. In FIG. 9, for example, such third coordinate is the angular coordinate 925, 926 of a cylindrical coordinate system. As illustrated in FIG. 9, the acoustic transducers 18 of the linear array have a finite thickness in the direction perpendicular to the reference axis 913. Therefore, these elements can generate acoustic fields with specific patterns characterized by an angular dependence. The angular dependence can affect both acoustic amplitude and/or the duration of the acoustic signal. Matching the angular dependence with acoustic signal measurements can allow one to determine the angular coordinates 925, 926.

Another approach for localization is to separate the acoustic sensors 28 (e.g., acoustic sensors 901, 902) mounted on the catheter 14 by a known distance. This distance provides an additional restriction that reduces ambiguity of localization. Consider, for example, a catheter with two sensors and a linear-array transducer shown in FIG. 9. The positions of the first and second acoustic sensors 901, 902 are described by axial coordinates 923, 924, radial distances 921, 922, and angles 925, 926, respectively. As discussed above, ToF measurements allow one to determine the axial coordinates 923, 924 and radial distances 921, 922, but not the angles 925, 926 due to the symmetry of the linear-array transducer. Use of a known distance between the first and second acoustic sensors 901, 902 allows one to assess the angle between the reference axis 913 and the direction of the catheter to determine the angles 925, 926.

It is noted that when the catheter 14 includes only one acoustic sensor 28, the direction of the catheter 14 is difficult to determine. The distance between the catheter 14 and the acoustic source 18 can be determined. Referring to FIG. 9 and assuming that the catheter 14 includes only the first acoustic sensor 901, the radial distance along radius line 921 and the axial coordinate 923 of the first acoustic sensor 901 (e.g., of the radius line 921) can be determined when at least two acoustic transducers 18 produce the acoustic signals 34. The angle 925 of the first acoustic sensor 901 (e.g., of the radius line 921) can be determined using, for example, the angular dependence of the acoustic signal on orientation of the acoustic transducer 18, as discussed above.

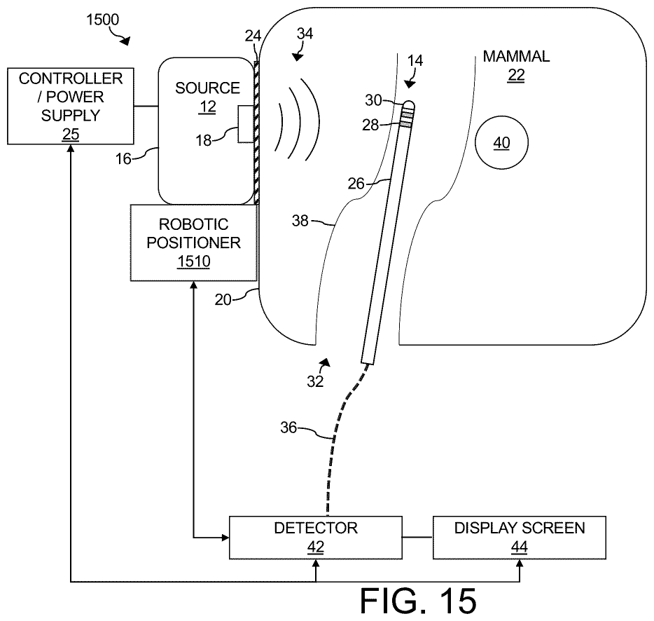

In some embodiments, the detector 42 can use the localization data to produce output signals that cause a robotic positioner 1510 (FIG. 15) to position the acoustic source 12 relative to the catheter, for example to align the acoustic source 12 with respect to the catheter 14. The robotic positioner 1510 and the detector 42 can communicate wirelessly or electrically or both.

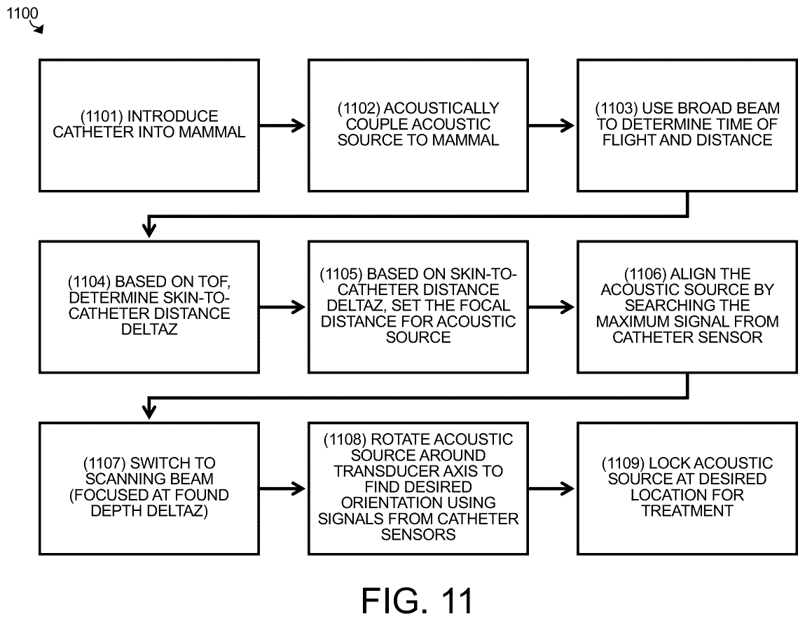

FIG. 11 is a flow chart of a method 1100 for localizing an acoustic source 12 and a catheter 14 with respect to each other according to another embodiment.

In step 1101, the catheter 14 is inserted into a natural or surgical opening 32 in a mammal.

In step 1102, the acoustic source 12 is acoustically coupled to a mammal 22 such as a human. The acoustic source 12 can be placed directly on the mammal 22 (e.g., on the skin 20 of the mammal 22) to acoustically couple the acoustic source 12 to the mammal 22. Alternatively, an acoustically transmitting media 24, such as water, a water cushion, an acoustic coupling oil, and/or an acoustic coupling gel, can be disposed between (e.g., in direct physical contact with) the acoustic source 12 and the skin 20 to improve acoustic transmission and acoustic coupling.

In step 1103, a broad beam of transcutaneous ultrasound is produced to determine the skin-to-catheter distance by measuring the ToF between the acoustic transducer(s) 18 and the acoustic sensor(s) 28. The ToF is used to determine the distance (e.g., delta Z) between the acoustic transducer(s) 18 on the skin and the acoustic sensor(s) 28 in step 1104. In step 1105, based on the measured distance (e.g., skin-to-catheter distance (e.g., delta Z)), the focal distance for the acoustic beam produced by the acoustic transducer(s) 18 is set.

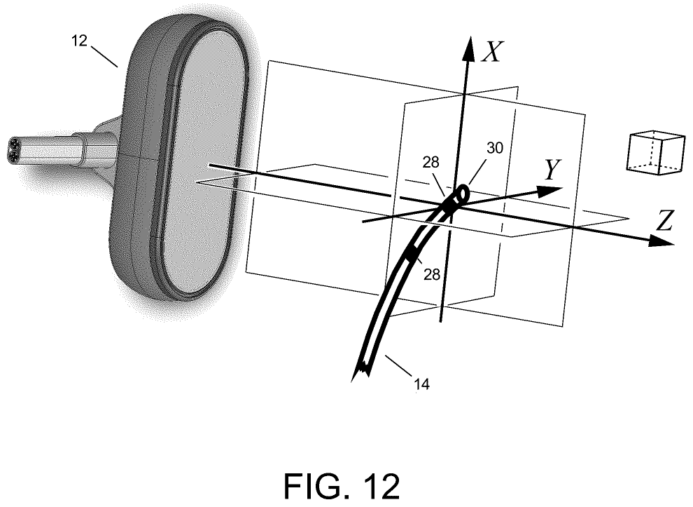

In step 1106, the acoustic transducer(s) 18 produce acoustic signals 34 that are focused at the focal distance determined in step 1104. When the acoustic source 12 only includes one acoustic transducer 18, the acoustic transducer 18 can include adjustable acoustic lenses and/or mechanical means to vary the focal distance. The acoustic source 12 is aligned with the catheter 14 by searching for a first maximum-amplitude signal measured by the acoustic sensor(s) 28 as the acoustic source 12 is moved or translated parallel to the Y-axis of the XYZ-coordinate system shown in FIG. 12. The first maximum-amplitude signal corresponds to a first localization, such as a first coordinate (e.g., with respect to the Y-axis) and a corresponding position of the acoustic source 12 (e.g., with respect to the Y-axis) on the mammal 22. In step 1107, once the acoustic source 12 is aligned with the acoustic sensor(s) 28, the acoustic field on the acoustic source 12 is switched to a sweeping focused beam (e.g., focused at distance/depth delta Z) while the acoustic source 12 remains at the position along the Y-axis that corresponds to the first maximum-amplitude signal. The focused beam can be swept by moving the acoustic source 12 parallel to the Y-axis and/or by varying the focal position electronically (e.g., by varying relative phases of the acoustic transducers 18 in a phased array). This will sweep the acoustic beam along or parallel to the X-axis of the XYZ-coordinate system shown in FIG. 12.

In step 1108, the acoustic source 12 is rotated about the transducer axis to find the orientation when signals on the acoustic sensor(s) 28 are maximized, indicating good alignment of the treatment head to the catheter in the ureter. In step 1109, the aligned acoustic source 12 is locked in place (e.g., at the position corresponding to the first maximum-amplitude signal) to perform a treatment procedure. The acoustic source 12 can be locked in place with a mechanical apparatus. It is noted that the localization occurs without imaging, including acoustic (e.g., ultrasound imaging) imaging.

FIG. 12 shows an example Cartesian coordinate system with three orthogonal coordinates X, Y, and Z. The X-axis is directed along the longitudinal axis of the acoustic source 12 (e.g., treatment head). The Z-axis is directed along the axis of symmetry of the acoustic source 12. The distal end 202 (FIG. 2) of the catheter 14 is located at distance Z from the acoustic source 12.

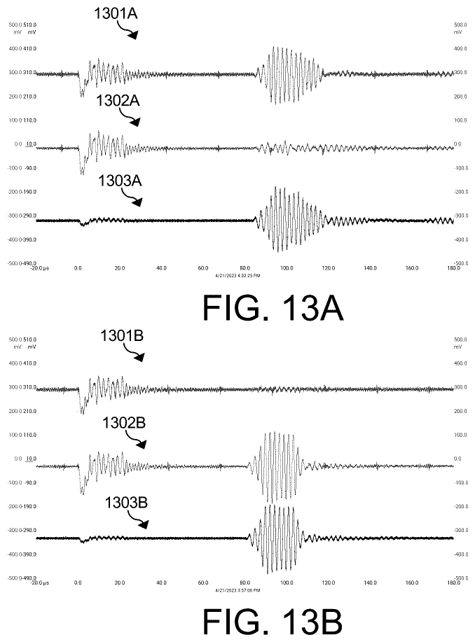

FIGS. 13A and 13B illustrate example traces 1301A, 1301B, 1302A, 1302B of two acoustic sensors 28 on a catheter 14 and their respective differential signals 1303A, 1303B demonstrating an alignment procedure for an acoustic source 12 to a catheter 14. One acoustic sensor 28 was mounted at about 3 cm from the catheter tip (traces 1301A, 1301B), the other near the tip (traces 1302A, 1302B). The catheter 14 was positioned along the X-axis of the acoustic source 12 (FIG. 12) at about 12 cm distance (Z-axis) from the acoustic source 12, corresponding to a ToF of about 80 μs. The acoustic source 12 emitted 10-cycle tone bursts at a center frequency of 450 kHz. The acoustic signals received by the acoustic sensors 28 were seen in a time window from 80-120 μs. In FIG. 13A, trace 1301A showed a greater amplitude of acoustic signal than that of trace 1302A detected by the transducer at the catheter tip. The displacement of the treatment head by 3 cm along the X-axis decreased the trace 1301B and increased the trace 1302B, indicating alignment of the treatment head 12 to the tip of catheter 14. The signal traces 1303A, 1303B show a differential signal, calculated by taking a difference between signals received by the two sensors 1301 and 1302, demonstrating a method to reduce EMI induced during emission of the acoustic source 12.

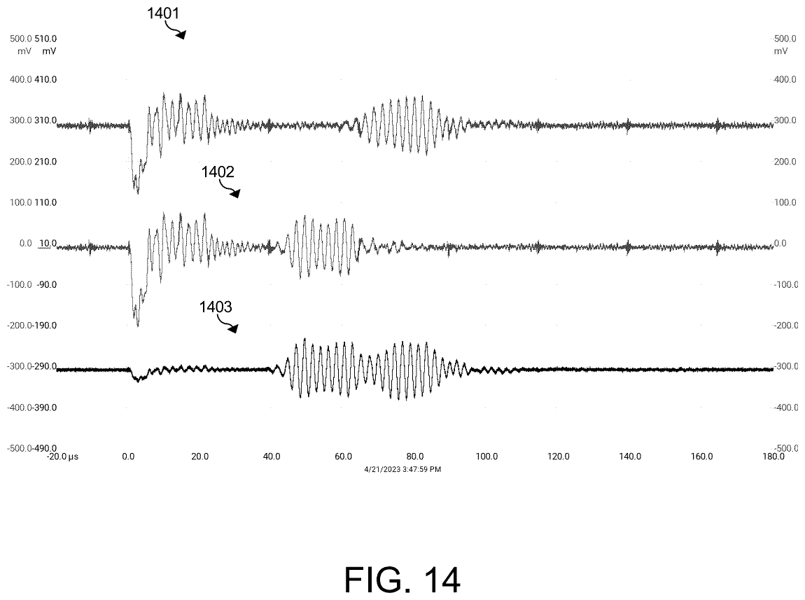

FIG. 14 shows signals from acoustic sensors 28 (traces 1401, 1402) mounted approximately 3 cm apart on a catheter 14 oriented along the acoustic axis Z of the acoustic source 12. The travel time of the acoustic signals over 3 cm distance was 20 μs. This time delay was observed between the traces 1401, 1402 indicating that the Z-axis of the treatment head was oriented such that the acoustic axis (Z-axis, FIG. 12) was along the length of the catheter 14. Furthermore, a shorter arrival time on the acoustic sensor 28 positioned closer to the tip 30 of the catheter 14 (trace 1402) compared to that of the acoustic sensor 28 positioned further from the tip 30 (trace 1401) indicates that the tip 30 of the catheter 14 was oriented toward the acoustic source 12. This orientation of the catheter 14 was confirmed visually, demonstrating the proposed alignment method. Trace 1403 represents a differential signal, demonstrating a method to reduce EMI induced simultaneously on both acoustic sensors 28 during the emission of the treatment head (about 0-20 μs). The acoustic source 12 emitted a 10-cycle tone burst with a center frequency of 450 kHz. The acoustic signals received by the acoustic sensors 28 were seen in time window from 40-100 μs, corresponding to the ToF for acoustic wave from the acoustic source 12 to the acoustic sensors 28.

A catheter 14 with acoustic sensors 28 can be used for guidance in medical procedures. The catheter 14 can be inserted either through a minute incision through the skin or through a natural orifice in the human body. Such applications include, for example, a biopsy of various organs or for percutaneous access in the kidney. For percutaneous access, the catheter 14 can be inserted through the ureter and can have a pre-set shape that will ensure a certain position of the catheter 14 in the kidney. For example, the catheter 14 can be designed such that it “prefers” a certain shape such that it tends to be positioned in the low pole of the kidney. The ultrasound source 12 can include a guiding fixture to guide a needle which can be used for the medical procedure such as for a biopsy or a percutaneous nephrolithotomy (PCNL). The guiding fixture can be adjustable so as to be oriented at the localized position of the catheter 14. The system can determine the depth for needle insertion, which can be the same as the measured depth or distance between the acoustic source 12 and the catheter 14, and can make sure that the needle avoids blood vessels, which is a typical concern and/or complication during the percutaneous access. An apparent advantage of all the potential applications of the catheter with acoustic sensors is avoiding or minimizing the use of harmful X-ray radiation that is currently used for guidance in these procedures.

Another unique advantage of a catheter 14 with acoustic sensors 28 is that this device provides absolute measurements of acoustic pressure in the treatment zone. This is important since the acoustic transmission is typically affected by various factors, such as, for example, mismatch of acoustic impedances at various interfaces, low acoustic transmission through ribs and bones, difference in attenuation of acoustic waves in different types of tissue, and presence of air or gas pockets along the acoustic path. Because of these factors, the actual acoustic pressure delivered to the treatment zone is typically unknown. This weakness can be addressed by using a catheter 14 with acoustic sensors 28.

FIG. 15 is a block diagram of a system 1500 for acoustically localizing an acoustic source 12 with respect to a catheter 14 according to another embodiment. System 1500 is the same as system 10 (FIG. 1) except that in system 1500 a robotic positioner 1510 is electromechanically coupled to the acoustic source 12. The detector 42 can send output control signals to the robotic positioner 1510 that cause the robotic positioner 1510 to position the acoustic source 12 in response to a localization of the acoustic source 12 and the catheter 14 relative to each other. For example, the robotic positioner 1510 can align the acoustic source 12 with respect to the catheter 14. The detector 42 and the robotic positioner 1510 can be connected electrically and/or wirelessly.

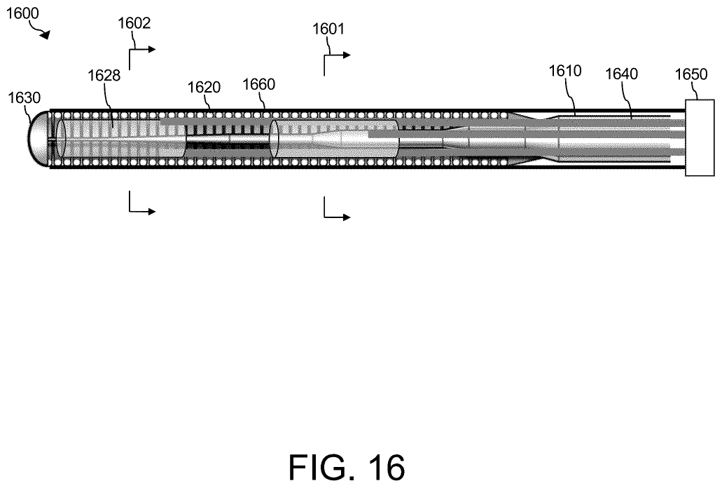

FIG. 16 is a partially transparent side view of a guidewire 1600 according to an embodiment. The guidewire 1600 includes a core 1610, an optional coil 1620, and one or more acoustic sensors 1628. The coil 1620 can be removed in some embodiments. The core 1610 and the coil 1620 are coaxial and extend from a proximal end to a distal end of the guidewire 1600. The acoustics sensor(s) 1628 can be located at predetermined position(s) from the distal end of the guidewire 1600 and/or from a tip 1630 at the distal end of the catheter 1600.

The acoustic sensor(s) 1628 is/are located between the core 1610 and the coil 1620 and are disposed about at least a portion of the core 1610. The acoustic sensor(s) 1628 are electrically connected to leads and/or wires 1640. The leads and/or wires 1640 can be electrically connected to one or more electrical connection points in a housing 1650 attached to a proximal end of the guidewire 1600. It is noted that the guidewire 1600 is not shown at scale and in practice the housing 1650 would be much further away from the tip 1630 and the acoustic sensor(s) 1628 than as illustrated.

The leads and/or wires 1640 can be disposed or embedded in a protective coating 1660 that covers the external surface of the guidewire 1600, such that the leads and/or wires 1640 extend parallel to the core 1610. The coil 1620 can be disposed or embedded in the protective coating 1660. In another embodiment, the core 1610 can be hollow and the leads and/or wires 1640 can pass through a channel in the core 1610.

The acoustic sensor(s) 1628 can be the same as the acoustic sensors 28. Additionally or alternatively, the housing 1650 can be the same as the housing 210.

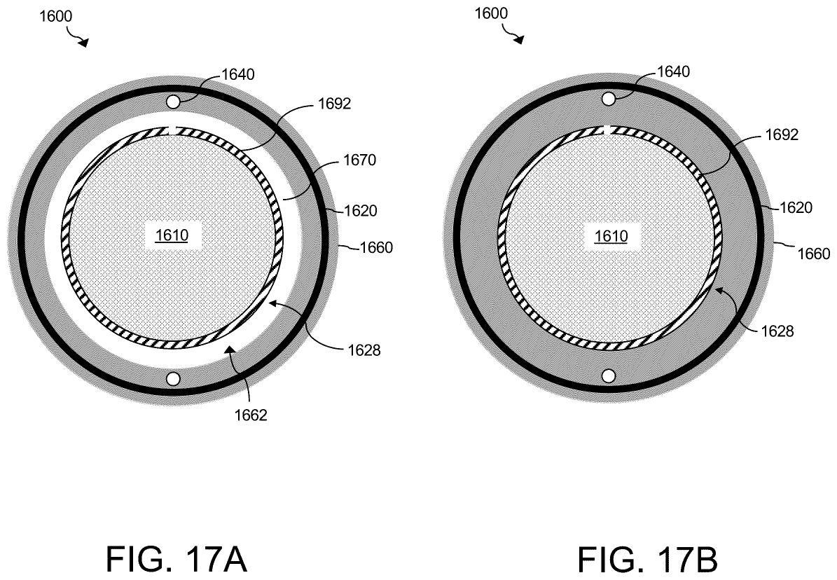

FIG. 17A is a cross section of the guidewire 1600 through plane 1601 in FIG. 16 according to an embodiment. The acoustic sensor 1628 includes a piezoelectric polymer film 1692 that is disposed about at least a portion of the circumference of the core 1610. The piezoelectric polymer film 1692 can be the same as the piezoelectric polymer film 392. In some embodiments, the acoustic sensor 1628 can include two piezoelectric polymer films (e.g., as illustrated in FIG. 6D).

To reduce or eliminate air gaps between the protective coating 1660 and the sensor 1628 (e.g., piezoelectric polymer film 1692), a gap 1662 between the protective coating 1660 and the sensor 1628 can be filled with one or more materials 1670 that have an acoustic impedance that matches (e.g., within about 20%) the impedance of bodily fluids and/or tissue, which have an acoustic impedance similar to that of water. The material(s) 1670 can be the same as the material(s) 600. In another embodiment, air gaps can be removed or reduced by the increasing the diameter of the core 1610 and/or by increasing the thickness of the protective coating 1660 so that the piezoelectric polymer film 1692 is in direct physical contact with the protective coating 1660 as well as with the core 1610, as illustrated in FIG. 17B.

A cross section of the guidewire 1600 through plane 1602 in FIG. 16 can be the same as the cross section illustrated in FIG. 17A or the cross section illustrated in FIG. 17B though the diameter of the core 1610 may be smaller in the cross section through plane 1602 than in the cross section through plane 1601.

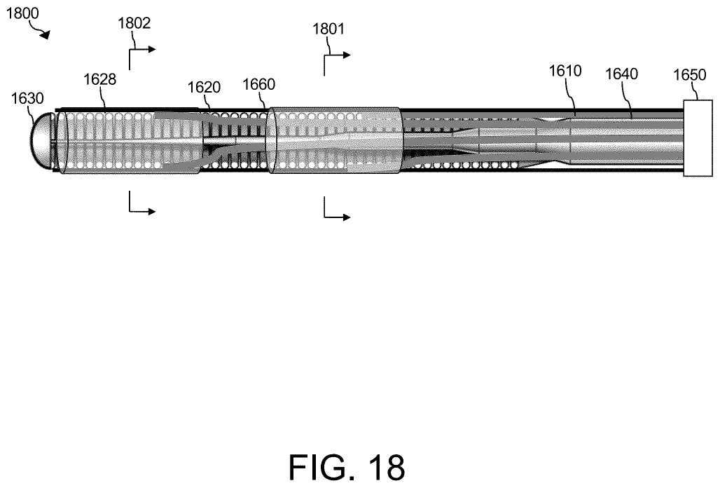

FIG. 18 is a partially transparent side view of a guidewire 1800 according to another embodiment. The guidewire 1800 is the same as the guidewire 1600 except that in the guidewire 1800 the acoustic sensor(s) 1628 is/are disposed on or in the protective coating 1660.

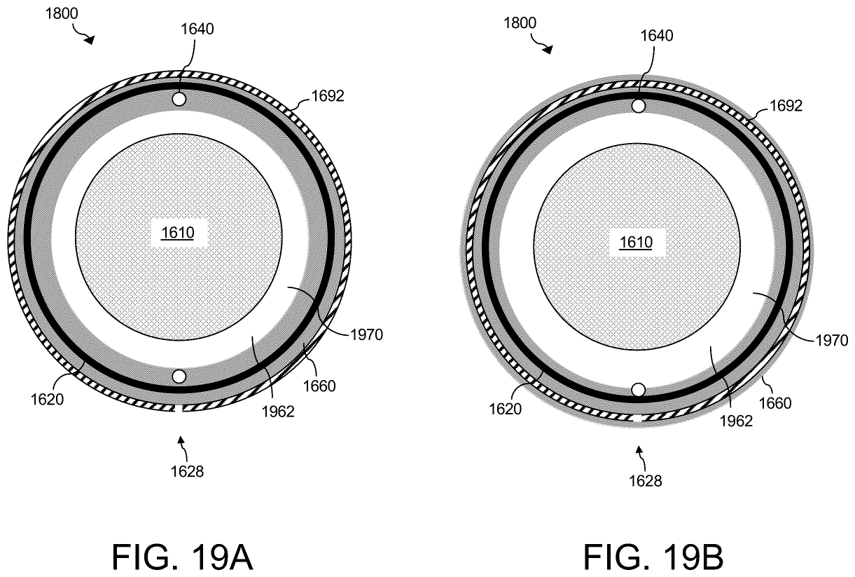

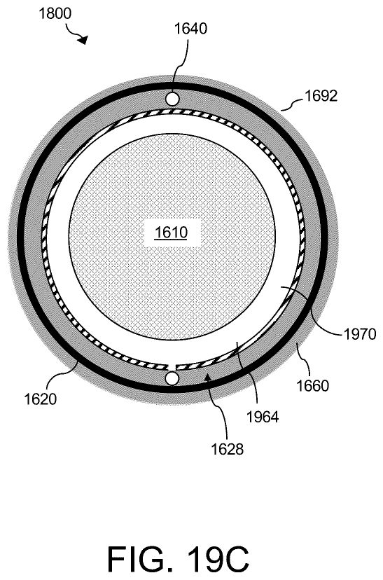

FIG. 19A is a cross section of the guidewire 1800 through plane 1801 in FIG. 18 according to an embodiment. The acoustic sensor 1628 includes a piezoelectric polymer film 1692 that is disposed on the external surface of the protective coating 1660. The piezoelectric polymer film is disposed about at least a portion of the circumference of the protective coating 1660. A gap 1962 between the protective coating 1660 and the core 1610 can be filled with one or more materials 1970 having a high acoustic impedance and/or that can provide an acoustically rigid interface to increase acoustic pressure at the acoustic sensor 1628. In some embodiments, the material(s) 1970 can be configured to absorb acoustic waves. Partial or complete absorption of acoustic waves (such as by an epoxy resin) can reduce or eliminate the reflection of acoustic waves, which may be desirable in some embodiments. The material(s) 1970 can be the same as the material(s) 610.

In some embodiments, the acoustic sensor 1628 can include two piezoelectric polymer films (e.g., as illustrated in FIG. 6D).

FIG. 19B is a cross section of the guidewire 1800 through plane 1801 in FIG. 18 according to another embodiment. The acoustic sensor 1628 includes a piezoelectric polymer film 1692 that is disposed or embedded in the protective coating 1660. The piezoelectric polymer film is disposed about at least a portion of the protective coating 1660. The gap 1962 can be filled with one or more materials 1970.

FIG. 19C is a cross section of the guidewire 1800 through plane 1801 in FIG. 18 according to another embodiment. The acoustic sensor 1628 includes a piezoelectric polymer film 1692 that is disposed on the internal surface of the protective coating 1660. The piezoelectric polymer film is disposed about at least a portion of the circumference of the inner diameter of the protective coating 1660. A gap 1964 between the piezoelectric polymer film 1692 and the core 1610 can be filled with one or more materials 1970.

A cross section of the guidewire 1800 through plane 1802 in FIG. 18 can be the same as any of the cross sections illustrated in FIG. 19A-C though the diameter of the core 1610 may be smaller in the cross section through plane 1602 than in the cross section through plane 1601.

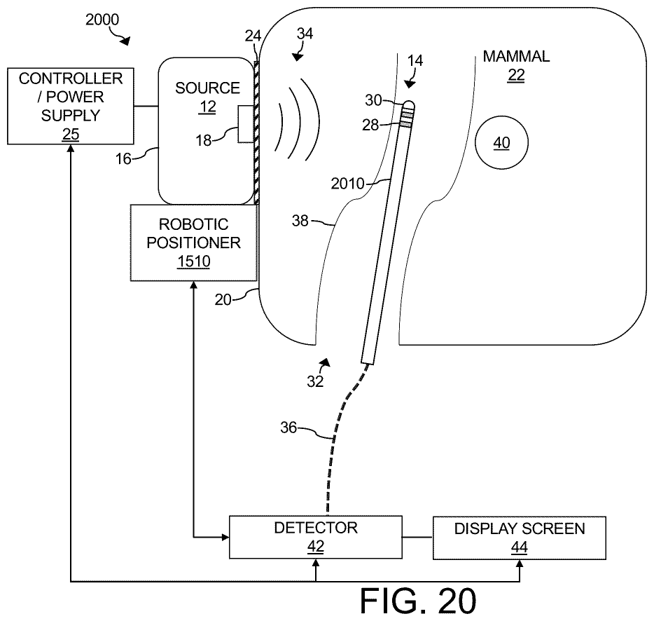

FIG. 20 is a block diagram of a system 2000 for acoustically localizing an acoustic source 12 with respect to a guidewire 2010 according to an embodiment. The system 2000 is the same as the system 10 and/or the system 1500 except that the catheter 14 in systems 10, 1500 is replaced with the guidewire 2010. The guidewire 2010 can be the same as the guidewire 1600 or the guidewire 1800.

An acoustic source 12 and a guidewire 2010 can be localized with respect to each other according to method 80 (FIG. 8) or according to method 1100 (FIG. 11) where the catheter 14 is replaced with a guidewire 2010.

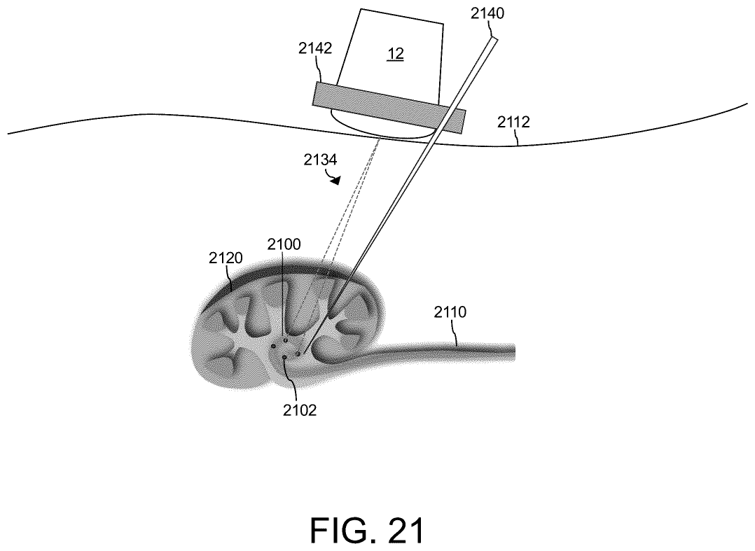

FIG. 21 shows an embodiment in which a medical device 2100 with multiple acoustic sensors 2102 is inserted through the ureter 2110 in the collecting system of the human kidney 2120. The medical device 2100 can be a catheter (e.g., catheter 14), a guidewire (e.g., guidewire 1600, 1800), or another medical device. The acoustic sensors 2102 can be the acoustic sensor(s) 28, the acoustic sensor(s) 1628, and/or the acoustic sensor(s) 1828.

When fully inserted, the medical device 2100 is configured to coil or uncoil, accepting a predetermined shape, e.g., forming a coil-like structure with a predetermined shape in the kidney 2120 or in another target location. The predetermined shape determines the relative position of the acoustic sensors 2102 to avoid the possible ambiguity of sensor position in the kidney 2120. An extracorporeal acoustic source 12, acoustically coupled to the patient's skin 2112, produces acoustic signal(s) 2134 which is received by the acoustic sensors 2102 to establish and/or determine the relative position of the acoustic source 12 and the acoustic sensors 2102 in the kidney 2120, for example using a detector 42. The acoustic signal(s) 2134 can be the same as the acoustic signals 34.

After the relative position of the acoustic source 12 and the acoustic sensors 2102 in the kidney 2120 is determined, a needle 2140 or similar medical device can now be inserted into the kidney 2120 (e.g., into the kidney calyx or a kidney wall), e.g., to perform a percutaneous access procedure, under the acoustic guidance of the acoustic source 12 and acoustic sensors 2102, as disclosed herein. The relative position and orientation (e.g., angle) of the needle 2140 is known relative to the acoustic source 12, for example using a needle guide or a needle bracket 2142, which can have an adjustable angle of insertion for the needle 2140. This approach is easy to perform and does not require special skills, such as a sonographic expert. This approach requires neither ultrasound imaging system nor harmful ionizing radiation imaging, such as X-ray imaging.

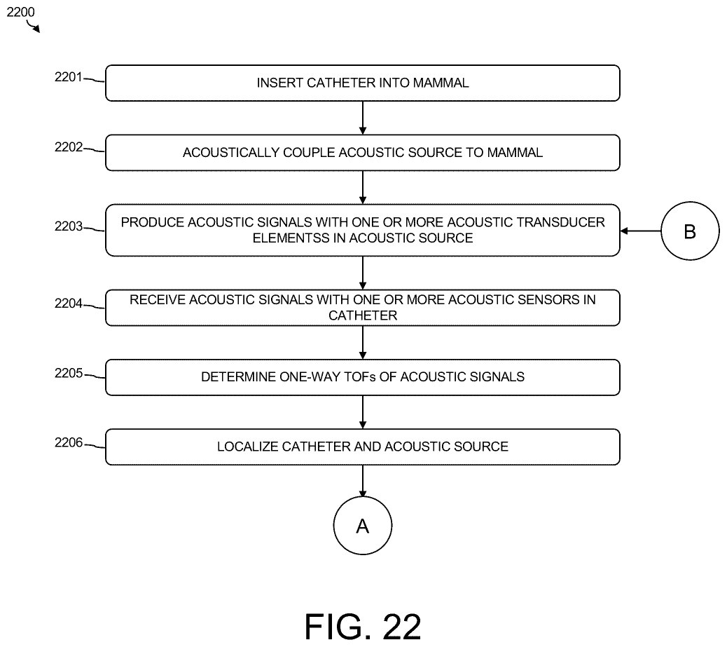

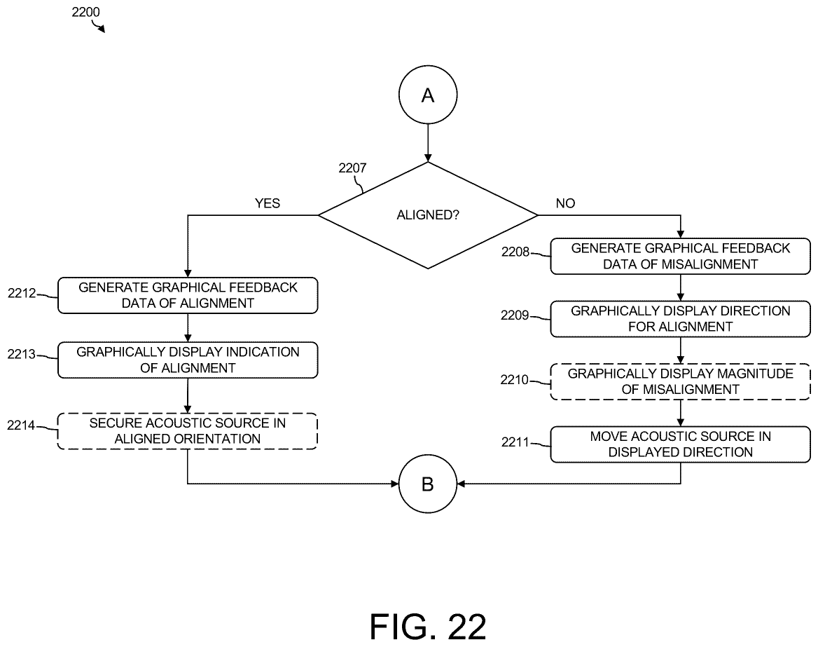

FIG. 22 is a flow chart of a method 2200 for localizing an acoustic source 12 and a catheter 14 with respect to each other according to one or more embodiments. Steps 2201-2205 are the same as or substantially the same as steps 801-805, respectively, of method 80 (FIG. 8).

In step 2206, the catheter 14 and acoustic source 12 are localized with respect to each other. The localization of the catheter 14 and acoustic source 12 with respect to each other can be determined using the ToF(s) of the acoustic signals 34 transmitted between the acoustic transducer(s) 18 and the acoustic sensor(s) 28, as determined and/or measured in step 2205. The catheter 14 and acoustic source 12 can be localized by the controller 25 and/or by the detector 42.

In step 2207 (via placeholder A), the controller 25 and/or the detector 42 determines whether the catheter 14 is aligned with the acoustic source 12. The catheter 14 is aligned with the acoustic source 12 when the acoustic sensor(s) 28 in the catheter 14 is/are axially positioned in the center or approximately in the center (e.g., within a predetermined range of the center such as within 5% of the center) of the transducer 18 in the acoustic source 12. Alternatively, the catheter 14 may be aligned with the acoustic source 12 when the acoustic sensor(s) 28 in the catheter 14 are positioned not at or near the center, but at another desirable location within the acoustic field produced by the acoustic source 12, such as a focal region of the acoustic source 12. Such a position may be selected to provide a desired proportion of the catheter 14 within the acoustic field—for example, to minimize the portion of the treatment zone occupied by the catheter 14 and/or to increase the portion of the treatment zone not occupied by the catheter 14, or vice versa. For instance, the catheter tip 30 may be aligned near the edge of the transducer 18, or within a predetermined distance from the edge or from a specified element of the linear-array transducer 18 (e.g., element 9), such as within 10% of that specified position, of the transducer 18 in the acoustic source 12. The number of degrees of freedom for alignment can vary depending on the number of acoustic sensors and/or the number of transducer elements in the transducer 18.

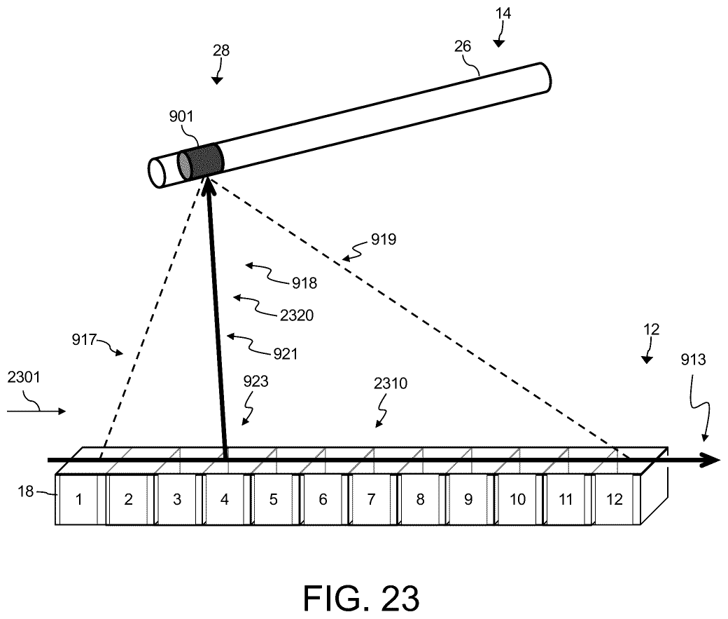

For example, when the ToF for an acoustic signal produced by acoustic element 12 of transducer 18 to reach acoustic sensor 901 is greater than the ToF for an acoustic signal produced by acoustic element 1 of transducer 18 to reach acoustic sensor 901, the catheter 14 and transducer 18 can be localized such that the distance 919 between acoustic element 12 and acoustic sensor 901 is greater than (e.g., outside a predetermined range of) the distance 917 between acoustic element 1 and acoustic sensor 901, as shown in FIG. 23. The distances 917, 919 can be used to determine an axial position or coordinate 923 of the acoustic sensor 901 relative to the transducer 18 and a corresponding radial distance 921 of the acoustic sensor 901 from the transducer 18 (e.g., from the reference axis 913). The position/coordinate 923 corresponds to an intersection of a radial axis 2320, along which the radial distance 921 is measured, and the reference axis 913. In this example, the axial coordinate 923 is positionally offset from the center 2310 of the transducer 18 located at the interface between elements 6 and 7. This may indicate that the transducer 18 is positionally offset (e.g., misaligned) in a first direction 2301 (e.g., to the left in FIG. 23) along the reference axis 913 with respect to the acoustic sensor 901. In one or more embodiments, the axial coordinate 923 can be compared with a predetermined range of the center 2310 of the transducer 18, which can be represented as an absolute range or a percentage range.

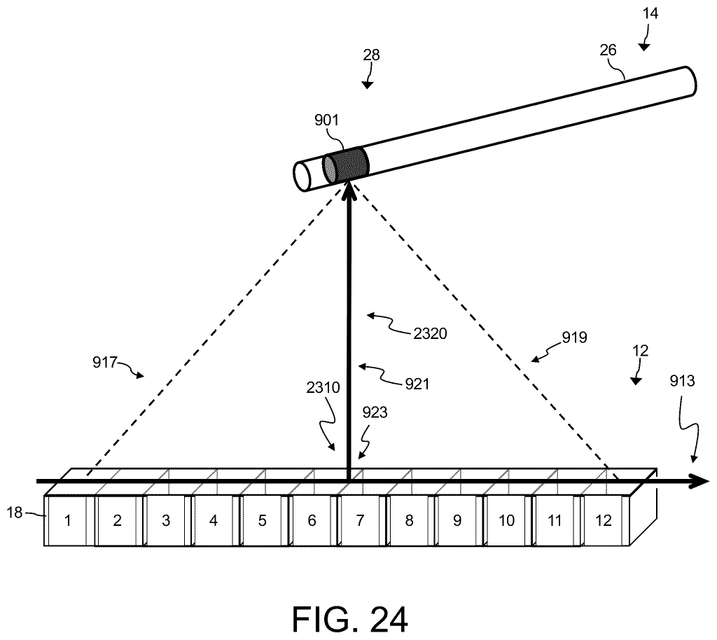

If/when the ToF for an acoustic signal produced by acoustic element 12 of transducer 18 to reach acoustic sensor 901 is equal or approximately equal to the ToF for an acoustic signal produced by acoustic element 1 of transducer 18 to reach acoustic sensor 901, the catheter 14 and transducer 18 can be localized such that the distance 919 between acoustic element 12 and acoustic sensor 901 is equal or approximately equal to (e.g., within a predetermined range of) the distance 917 between acoustic element 1 and acoustic sensor 901, as shown in FIG. 24. The distances 917, 919 can be used to determine an axial coordinate 923 of the acoustic sensor 901 and a corresponding radial distance 921 of the acoustic sensor 901 from the reference axis 913. In this example, the axial coordinate 923 is aligned with (e.g., within a predetermined range of the center 2310 of the transducer 18) the center 2310 of the transducer 18 at the interface between acoustic elements 6 and 7. This may indicate that the transducer 18 is axially aligned with respect to the acoustic sensor 901 (i.e., step 2207=yes).

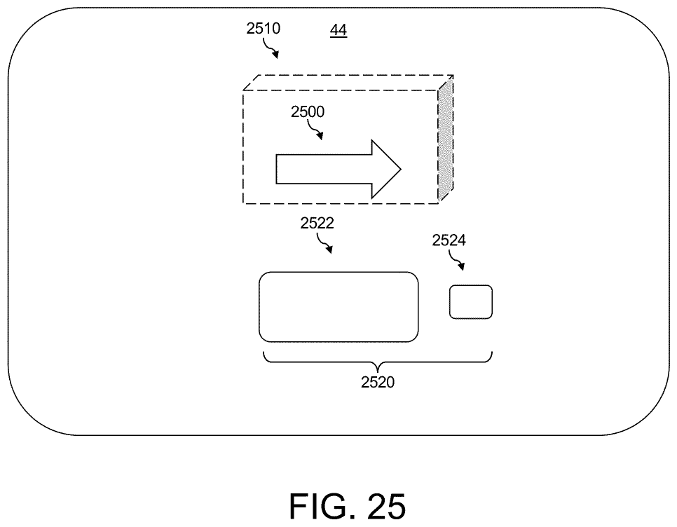

If/when the acoustic sensor 901 is positionally axially offset from the center 2310 of the transducer 18 along reference axis 913 in a first direction 2301 (i.e., step 2207=no), in step 2208 the controller 25 and/or detector 42 can generate graphical feedback data of axial misalignment that can be displayed on the display screen 44 in step 2209 and/or in optional step 2210, for example as shown in FIG. 25. The graphical feedback data can represent an arrow 2500 that indicates the direction to move (e.g., translate) the acoustic source 12 across the patient's body to align the transducer 18 relative to the acoustic sensor 901. The arrow 2500 can be overlaid on or displayed next to a graphical representation 2510 of the acoustic source 12 to clarify the object to be moved (the acoustic source 12) and the direction of movement. In this example, the arrow 2500 is pointed in a second direction that is opposite to the first direction 2301. Additionally or alternatively, the graphical feedback data can represent a graphical alignment representation 2520 that indicates a position of the acoustic sensor 901, as represented by icon 2522, relative to a target position in which the acoustic sensor 901 (or more generally, the distal end of the catheter 14) would be positionally aligned with the transducer 14 (or more generally, with the acoustic source 12), as represented by icon 2524. Alternatively, the icon 2522 can represent the current position of the transducer 18 and icon 2524 can represent a target location for the transducer 18, relative to its current position. The transducer 18 would be aligned with the acoustic sensor 901 when the transducer 18 is positioned at the target location as indicated by the icon 2524.

The difference in the relative positions of the icons 2522, 2524 can represent the direction to move (e.g., translate) the transducer 14 relative to the acoustic sensor 901 and/or the magnitude of misalignment, and thus the magnitude of the movement (e.g., in the direction of arrow 2550 and/or according to the relative positions of the icons 2522, 2524), to position the transducer 14 in alignment with the acoustic sensor 901.

In step 2211, the acoustic source 12 is moved in the displayed direction (e.g., according to the direction of the arrow 2500 and/or as indicated by the relative positions of the icons 2522, 2524. After step 2211, the method 2200 returns to step 2203 (via placeholder B) in a loop where additional acoustic signals are produced and measured. In one or more embodiments, the method 2200 can return to step 2203 even if the acoustic source 12 is not moved in step 2211 so as to iteratively and/or continually update the relative position of the catheter 14 and the acoustic source 12 in real time.

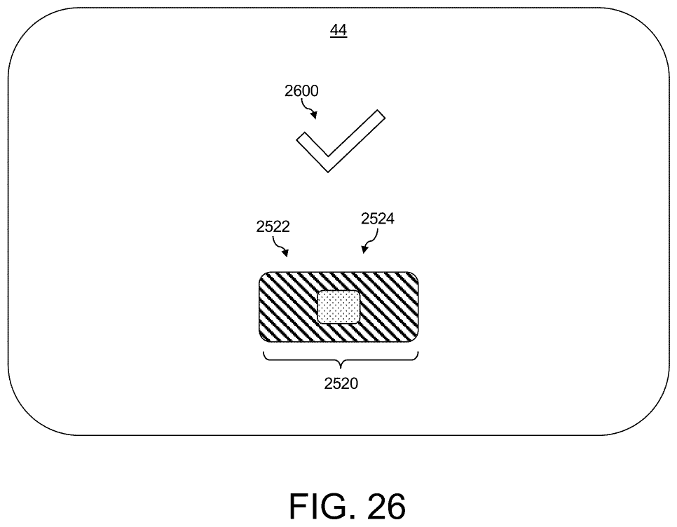

If/when the acoustic sensor 901 is positionally axially aligned with the center 2310 of the transducer 18 (i.e., step 2207=yes), for example after repositioning the transducer 14 according to the graphical display (e.g., in step(s) 2209 and optional 2210), in step 2212 the controller 25 and/or detector 42 can generate graphical feedback data of alignment that can be displayed on the display screen 44 in step 2213. In one or more embodiments, the state of the icon 2522 and/or state of the icon 2524 can be different when the transducer 14 is located at the target position in which the acoustic sensor 901 is positionally axially aligned with transducer 14 in step 2208, for example as shown in FIG. 26. Additionally or alternatively, the arrow 2500 can be replaced with a check mark 2600 or another symbol that can indicate that the acoustic sensor 901 is aligned with acoustic source 12, as shown in FIG. 26.

In optional step 2214, the acoustic source 12 can be secured in the aligned orientation, relative to the acoustic sensor 901, using a positioning accessory, such as a clamp.

After step optional 2214, the method 2200 can returns to step 2203 (via placeholder B) in a loop where additional acoustic signals are produced and measured. In one or more embodiments, the method 2200 can return to step 2203 so as to iteratively and/or continually update the relative position of the catheter 14 and the acoustic source 12 in real time, for example while the acoustic source 12 is being secured in optional step 2214.

When the catheter 14 includes a second acoustic sensor 902, the ToF for an acoustic signal produced by acoustic element 12 of transducer 18 to reach acoustic sensor 902 can be compared with the ToF for an acoustic signal produced by acoustic element 1 of transducer 18 to reach acoustic sensor 902. The difference in the TOFs for an acoustic signal to reach the second acoustic sensor 902 can be compared with the difference in the TOFs for an acoustic signal to reach the first acoustic sensor 901 to localize the first and second acoustic sensors 901, 902 (e.g., the distal end of the catheter 14) with respect to the transducer 14 (e.g., the acoustic source 12) in at least two dimensions. Localization with respect to a third dimension can be determined using a known distance between the first and second acoustic sensors 901, 902.

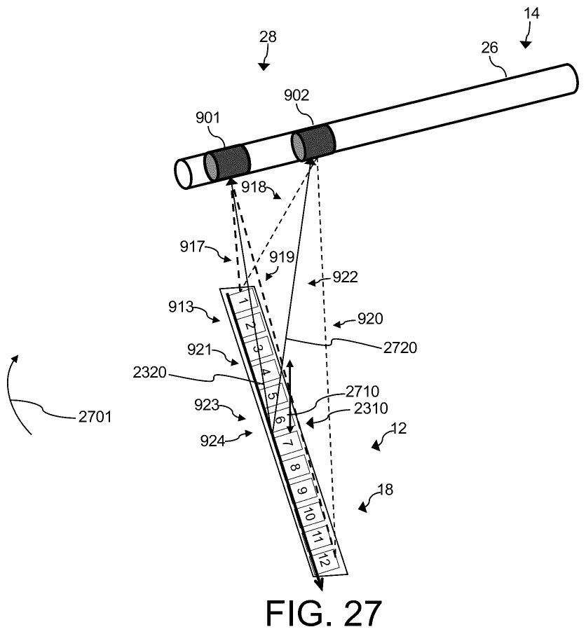

Referring to FIG. 27, the catheter 14 is positioned such that the ToF for an acoustic signal produced by acoustic element 12 of transducer 18 to reach the first acoustic sensor 901 is equal to or approximately equal to (e.g., within a predetermined range of) the ToF for an acoustic signal produced by acoustic element 1 of transducer 18 to reach the first acoustic sensor 901, and the ToF for an acoustic signal produced by acoustic element 12 of transducer 18 to reach the second acoustic sensor 902 is equal or approximately equal to (e.g., within a predetermined range of) the ToF for an acoustic signal produced by acoustic element 1 of transducer 18 to reach the second acoustic sensor 902. These ToF measurements indicate that (a) the distance 919 between acoustic element 12 and the first acoustic sensor 901 is equal or approximately equal to (e.g., within a predetermined range of) the distance 917 between acoustic element 1 and the first acoustic sensor 901 and (b) the distance 920 between acoustic element 12 and the second acoustic sensor 902 is equal or approximately equal to (e.g., within a predetermined range of) the distance 918 between acoustic element 1 and the second acoustic sensor 902. The distances 917, 919 can be used to determine an axial coordinate 923 of the first acoustic sensor 901, a radial distance 921 of the first acoustic sensor 901 from the reference axis 913, an axial coordinate 924 of the second acoustic sensor 902, and a radial distance 922 of the second acoustic sensor 901 from the reference axis 913. The radial distances 921, 922 are measured along respective radial axes 2320, 2720. The axial positions/coordinates corresponds to the intersection of the respective radial axes 2320, 2720 and the reference axis 913.

In this example, the axial coordinates 923, 924 are located at or near (e.g., within a predetermined range of) the same spatial position on the transducer 18 with respect to the reference axis 913. The reference axis 913 can alternately be referred to as a longitudinal axis of the transducer 18. In FIG. 27, the axial coordinates 923, 924 are located at or near the center 2310 of the transducer 18 between acoustic elements 6 and 7. Additionally, the radial distances 921, 922 are equal or approximately equal to (e.g., within a predetermined range of) each other. This information indicates that the transducer 18 is rotationally offset (e.g., misaligned), relative to the acoustic sensors 901, 902, in a first rotational direction 2701 about a second reference axis 2710 that is orthogonal to the reference axis 913 and that passes through the center 2310. For example, this information indicates that the shaft 26 of the catheter 14 is not extending parallel to (in this example the shaft 26 is orthogonal to) the reference axis 913. Axis 2710 is parallel to radial axes 2320, 2720. Axis 2710 can alternately be referred to as an acoustic axis of the transducer 18.

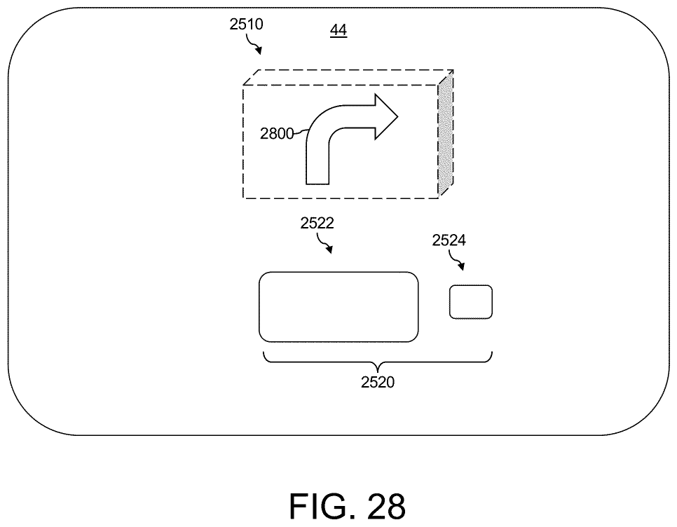

If/when the transducer 18 is rotational misaligned in a first rotational direction 2701 (e.g., step 2207=no), the controller 25 and/or detector 42 can generate graphical feedback data representing the rotational misalignment (e.g., in step 2208) that can be displayed on the display screen 44 in step(s) 2209 and optional step 2210, for example as shown in FIG. 28. The graphical feedback data can represent an arrow 2800 that indicates the direction to rotate the acoustic source 12 on the patient's body to align the transducer 18 relative to the acoustic sensors 901, 902. In this example, the arrow 2800 is pointed in a second rotational direction that is opposite to the first direction 2701. The arrow 2800 can be overlaid on or displayed next to a graphical representation 2510 of the acoustic source 12 to clarify the object to be rotated (the acoustic source) and the rotational orientation/direction of the arrow 2800 relative to the acoustic source 12. The graphical feedback data can also include a graphical alignment representation 2520, as discussed above, that can represent the direction to rotate and/or the magnitude of misalignment). The difference in the relative positions of the icons 2522, 2524 can represent the direction to move (e.g., rotate) the transducer 14 relative to the acoustic sensors 901, 902 and/or the magnitude of misalignment, and thus the magnitude of the movement (e.g., in the direction of arrow 2800 and/or according to the relative positions of the icons 2522, 2524), to position the transducer 14 in alignment with the acoustic sensors 901, 902.

In step 2211, the acoustic source 12 is moved in the displayed direction (e.g., rotated according to the direction of the arrow 2800) and/or as indicated by the relative positions of the icons 2522, 2524. After step 2211, the method 2200 returns to step 2203 (via placeholder B) in a loop where additional acoustic signals are produced and measured. In one or more embodiments, the method 2200 can return to step 2203 even if the acoustic source 12 is not moved in step 2211 so as to iteratively and/or continually update the relative position of the catheter 14 and the acoustic source 12 in real time.

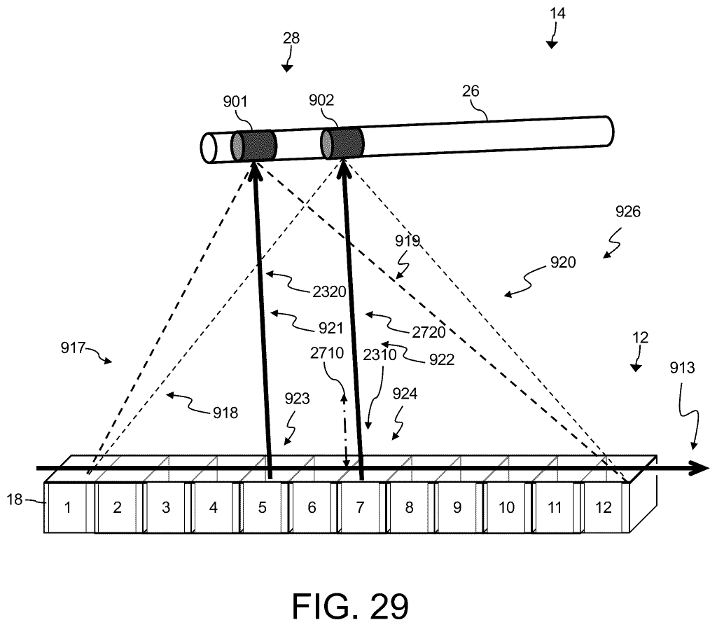

FIG. 29 shows the transducer 18 aligned with the first and second acoustic sensors 901, 902 after the acoustic source 12 is rotated in a second rotational direction (e.g., compared to the orientation of the acoustic source 12 in FIG. 27) such that the transducer 18 and the first and second acoustic sensor 901, 902 are rotationally aligned with respect to axis 2710. As shown in FIG. 29, the axial coordinates 923, 924 of the acoustic sensors 901, 902, respectively, are located at acoustic elements 5 and 7, respectively, of the transducer 12 indicating spatial alignment at or near (e.g., within a predetermined range of) the center 2310 of the transducer 12. The difference in the axial coordinates 923, 924 also indicates that the shaft 26 of the catheter 14 is parallel or approximately parallel (e.g., within a predetermined range of) to the reference axis 913.

When the transducer 18 and the first and second acoustic sensor 901, 902 are rotationally aligned with respect to axis 2710, in step 2212 the controller 25 and/or detector 42 can generate graphical feedback data of alignment (e.g., rotational and/or axial alignment) that can be displayed on the display screen 44 in step 2213, for example as shown in FIG. 26.

When the transducer 18 and the first and second acoustic sensor 901, 902 are rotationally aligned with respect to axis 2710, in step 2212 the controller 25 and/or detector 42 can generate graphical feedback data of alignment (e.g., rotational and/or axial alignment) that can be displayed on the display screen 44 in step 2213, for example as shown in FIG. 26.

In optional step 2214, the acoustic source 12 can be secured in the aligned orientation, relative to the acoustic sensor 901, using a positioning accessory, such as a clamp.

After step optional 2214, the method 2200 can returns to step 2203 (via placeholder B) in a loop where additional acoustic signals are produced and measured. In one or more embodiments, the method 2200 can return to step 2203 so as to iteratively and/or continually update the relative position of the catheter 14 and the acoustic source 12 in real time, for example while the acoustic source 12 is being secured in optional step 2214.

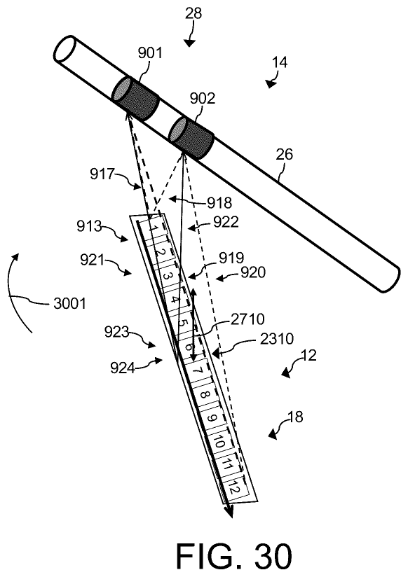

FIG. 30 shows an example where the catheter 14 is positioned relative to the acoustic source 12 such that the axial positions/coordinates 923, 924 of the first and second acoustic sensors 901, 902 are axially aligned with the center of the transducer 18 but the first radial distance 921 is larger than the second radial distance 922. This information indicates that the transducer 18 is rotationally offset (e.g., misaligned) in a first rotational direction 3001 about the reference axis 913 (e.g., step 2207=no) relative to the acoustic sensors 901, 902.

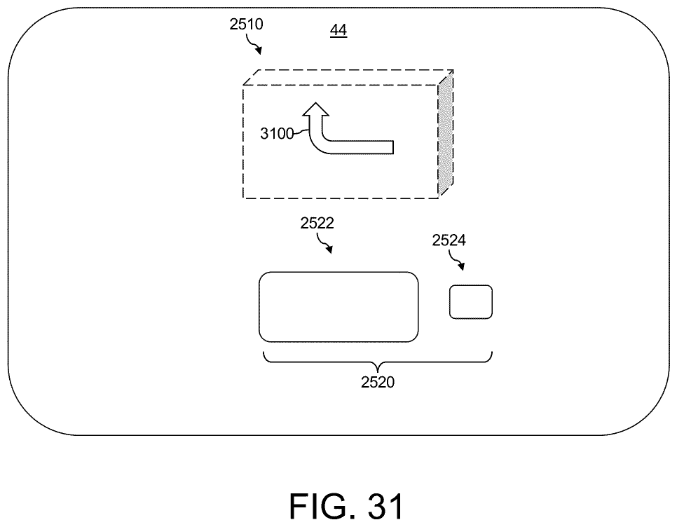

If/when the transducer 18 is offset (e.g., misaligned) in a first rotational direction 3001, the controller 25 and/or detector 42 can generate graphical feedback data (e.g., in step 2208) that can be displayed on the display screen 44 in step(s) 2209 and optional step 2210, for example as shown in FIG. 31. The graphical feedback data can represent an arrow 3100 that indicates the direction to rotate the acoustic source 12 on the patient's body. In this example, the arrow 3100 is pointed in a second rotational direction that is opposite to the first direction 3001. The arrow 3100 can be overlaid on or displayed next to a graphical representation 2510 of the acoustic source 12 to clarify the rotational orientation of the arrow 3100 relative to the acoustic source 12. The graphical feedback data can also include a graphical alignment representation 2520, as discussed above. The difference in the relative positions of the icons 2522, 2524 can represent the direction to move (e.g., rotate) the transducer 14 relative to the acoustic sensors 901, 902 and/or the magnitude of misalignment, and thus the magnitude of the movement (e.g., in the direction of arrow 3100 and/or according to the relative positions of the icons 2522, 2524), to position the transducer 14 in alignment with the acoustic sensors 901, 902.

In step 2211, the acoustic source 12 is moved in the displayed direction (e.g., rotated according to the direction of the arrow 3100) and/or as indicated by the relative positions of the icons 2522, 2524. After step 2211, the method 2200 returns to step 2203 (via placeholder B) in a loop where additional acoustic signals are produced and measured. In one or more embodiments, the method 2200 can return to step 2203 even if the acoustic source 12 is not moved in step 2211 so as to iteratively and/or continually update the relative position of the catheter 14 and the acoustic source 12 in real time.

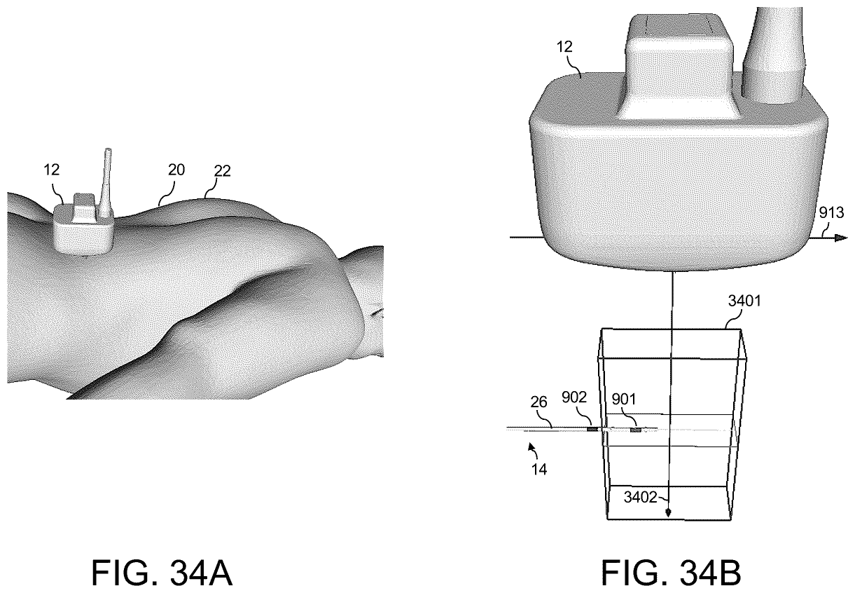

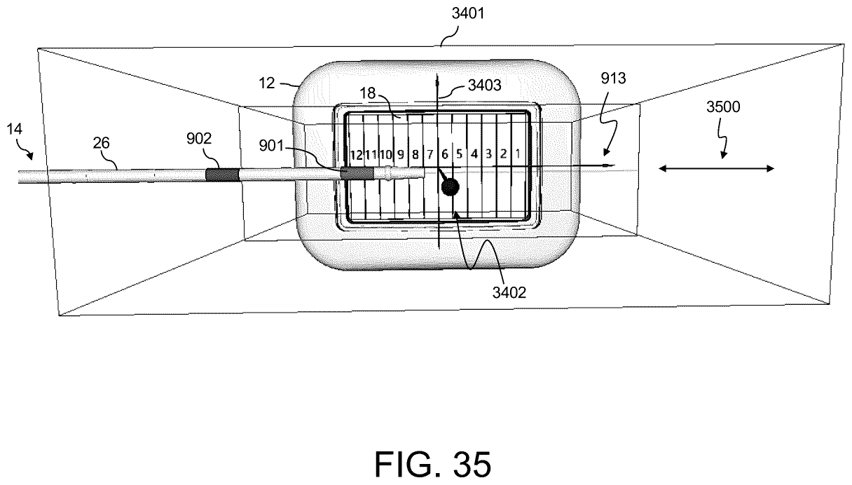

After the transducer 18 is rotated in a second rotational direction (e.g., compared to the orientation of the acoustic source 12 in FIG. 30), the transducer 18 is rotationally aligned with the first and second acoustic sensors 901, 902 with respect to the reference axis 913, for example as shown in FIGS. 34 and 35. When the transducer 18 and the first and second acoustic sensor 901, 902 are rotationally aligned with respect to the reference axis 913, in step 2212 the controller 25 and/or detector 42 can generate graphical feedback data of alignment that can be displayed on the display screen 44 in step 2213, for example as shown in FIG. 26.

In optional step 2214, the acoustic source 12 can be secured in the aligned orientation, relative to the acoustic sensor 901, using a positioning accessory, such as a clamp.

After step optional 2214, the method 2200 can returns to step 2203 (via placeholder B) in a loop where additional acoustic signals are produced and measured. In one or more embodiments, the method 2200 can return to step 2203 so as to iteratively and/or continually update the relative position of the catheter 14 and the acoustic source 12 in real time, for example while the acoustic source 12 is being secured in optional step 2214.

It is noted that while the foregoing examples describe localization of the acoustic source 12 (e.g., transducer 18) relative to the catheter 14 (e.g., sensor 901, 902) using ToF measurements for acoustic signals produced by acoustic elements 1 and 12 of the transducer 18, ToF measurements for acoustic signals produced by one or more other acoustic elements (e.g., 2-11) can be used instead of or in addition to those produced by acoustic elements 1 and 12. For example, ToF measurements for acoustic signals produced by acoustic element(s) 2 and/or 11 can be used instead of or in addition to the ToF measurements for the acoustic signals produced by acoustic elements 1 and 12. In one or more embodiments, the acoustic source 12 (e.g., transducer 18) can be localized relative to the catheter 14 (e.g., sensor 901, 902) using ToF measurements for acoustic signals produced by all acoustic elements (e.g., 1-12). Each acoustic element can produce a respective acoustic signal separately in time such that the acoustic signals are produced by each element sequentially, which can be repeated in a loop.

In one or more embodiments, the radial distance(s) 921, 922 and/or an average of the distances 921, 922 can be displayed on the display 44, for example in combination with the graphical indication of alignment displayed in step 2213 and/or with the graphical indication of misalignment in step 2209.

In one or more embodiments, the controller 25 and/or detector 42 can measure the amplitude of the acoustic signals received by the acoustic sensor(s) 901 and/or 902 and can compare the measured amplitude with a predetermined amplitude that can represent an expected amplitude of the acoustic signals when the transducer 18 is aligned with the acoustic sensor(s) 901 and/or 902, for example be determined according to modeling. A difference, a ratio, and/or a percentage of the measured amplitude with respect to the predetermined amplitude can be displayed on the display 44, for example in combination with the graphical indication of alignment displayed in step 2213 and/or with the graphical indication of misalignment in step 2209.

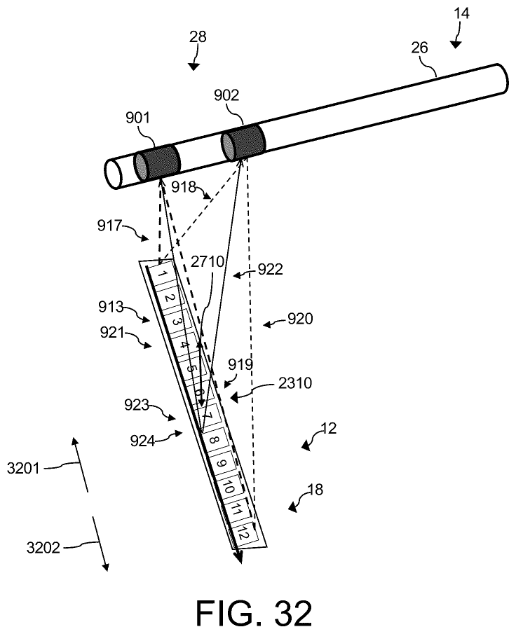

Referring to FIG. 32, the catheter 14 relative to the acoustic source such that that the axial position 923 of the first acoustic sensor 901 and the axial position 924 of the second acoustic sensor 902 are the same or approximately the same (e.g., within a predetermined range of) as each other, but the axial positions 923, 924 are not axially aligned with (e.g., within a predetermined range of) the center 2310 of the transducer 18. This indicates that the that the transducer 18 is axially offset (e.g., axially misaligned), in a first direction 3201 parallel to the reference axis 913, relative to the acoustic sensors 901, 902 and that the transducer 18 is rotationally aligned, with respect to the second reference axis 2710 relative to the acoustic sensors 901, 902. In addition, the respective radial distances 921, 922 of the acoustic sensors 901, 902 are equal or approximately equal (e.g., within a predetermined range of) indicating that the transducer 18 is rotationally aligned, with respect to the reference axis 913, relative to the acoustic sensors 901, 902.

When axial misalignment is detected, for example as shown in FIG. 32, graphical feedback data of axial misalignment can be displayed on the display screen 44 in step 2209 and/or in optional step 2210, for example as shown in FIG. 25. After the transducer 18 is moved axially in a second direction 3202 parallel to the reference axis and opposite to the first direction 3201, the transducer 18 is rotationally aligned with the first and second acoustic sensors 901, 902 with respect to the reference axis 913, for example as shown in FIG. 30.

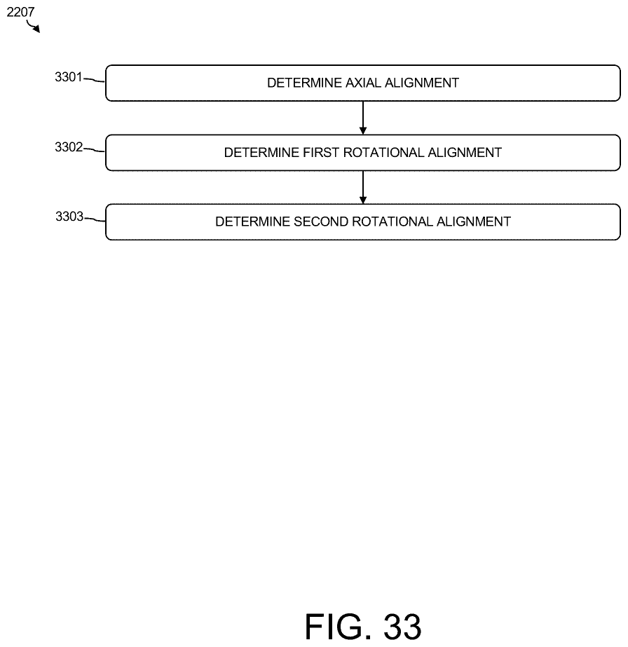

FIG. 33 is a flow chart of step 2207 of method 2200 according to one or more embodiments. In one or more embodiments, determining whether the catheter 14 is aligned with the acoustic source 12 in step 2207 includes determining an axial alignment of the transducer 18 with respect to the acoustic sensor(s) 901 and/or 902 in step 3301, determining a first rotational alignment of the transducer 18, relative to the reference axis 913, with respect to the acoustic sensor(s) 901 and/or 902 in step 3302, and/or determining a second rotational alignment of the transducer 18, relative to the second reference axis 2710, with respect to the acoustic sensor(s) 901 and/or 902 in step 3303, as described herein.

FIGS. 34A, 34B, and 35-39 illustrate one or more embodiments corresponding to the flowchart disclosed in FIG. 33. FIG. 34A shows an acoustic source 12 coupled to the skin 20 of a human mammal 22 in the lower back region according to one or more embodiments. FIG. 34B is a perspective view of the acoustic source 12, illustrating a longitudinal axis 913, an acoustic axis 3402, and a treatment region represented as a cuboid 3401, according to one or more embodiments. The objective is to align the treatment region 3401 with the spatial position of the catheter 14, inserted into the body of the human mammal 22 and not visible with the naked eye, using acoustic signals received by acoustic sensors 901 and 902 mounted on the catheter shaft 26.

FIG. 35 shows a front perspective view of the transducer 18 as seen from the direction of the catheter 14 according to one or more embodiments. In this example, the catheter sensors 901 and 902 are positioned in front of the acoustic source 12, depicted as a linear array of piezoelectric transducer elements 18. The acoustic source 12 is spatially aligned with the catheter 14 such that the longitudinal axis 913 of the linear array transducer 18 is parallel to the shaft 26 of the catheter 14 (e.g., the shaft 26 extends parallel to the longitudinal axis 913), and element 9 of the linear array transducer 18 is spatially aligned with the acoustic sensor 901. The shaft 26 extends along or parallel to a shaft axis 3500. The shaft axis 3500 is parallel to the longitudinal axis 913 in FIG. 35. It is noted that element 9 appears laterally offset from (e.g., to the right of) the acoustic sensor 901 due to the perspective view of FIG. 35.

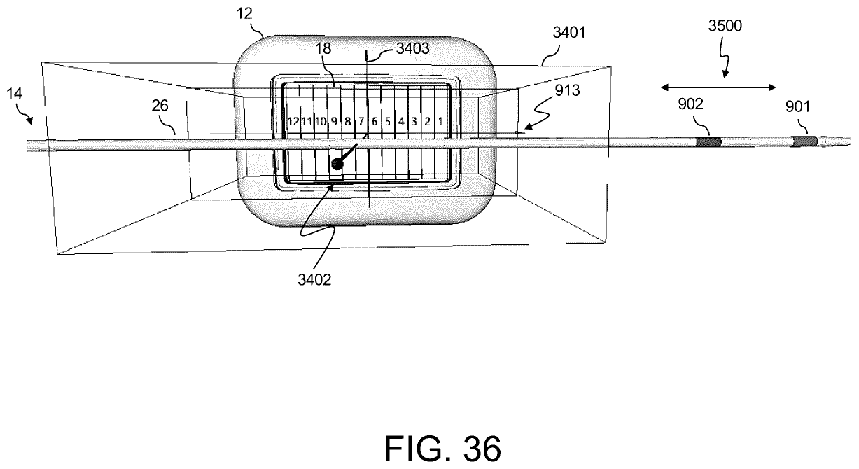

This or another desired alignment can be achieved by following the flow chart described in FIG. 33, beginning with the axial movement of an initially misaligned acoustic source 12 along its longitudinal axis 913. FIG. 36 is another front perspective view of the transducer 18 as seen from the direction of the catheter 14 according to one or more embodiments. FIG. 36 illustrates the acoustic source 12 in a state of initial axial misalignment with the catheter 14 such that the acoustic sensors 901 and 902 are located outside of the treatment region 3401 along the longitudinal axis 913. To transition the acoustic source 12 to an aligned state (e.g., as shown in FIG. 29), the acoustic source 12 can be moved laterally, parallel to longitudinal axis 913 and/or parallel to shaft axis 3500, towards the acoustic sensors 901, 902 (e.g., to the right in FIG. 36).

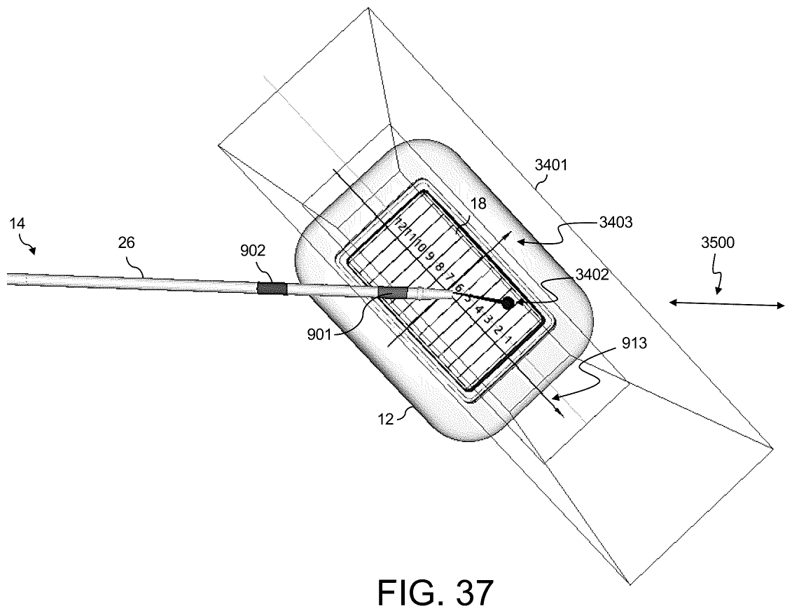

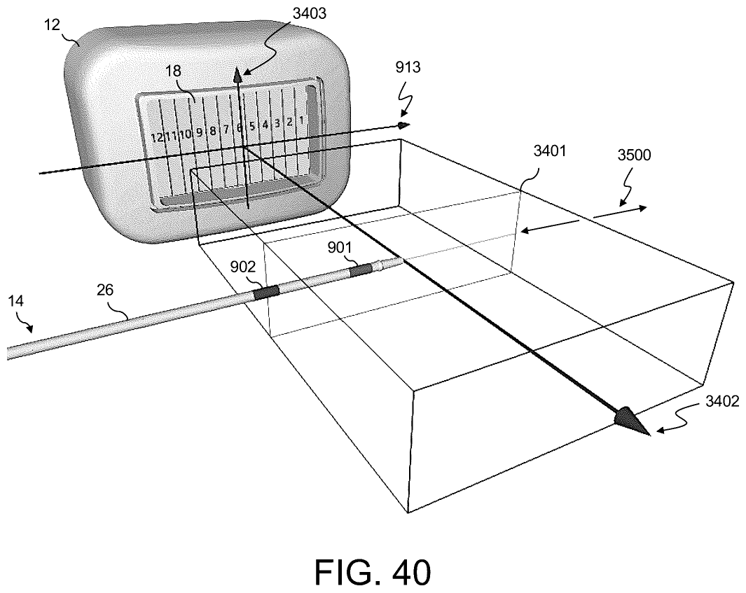

FIG. 37 is another front perspective view of the transducer 18 as seen from the direction of the catheter 14 according to one or more embodiments. FIG. 37 illustrates a rotational misalignment condition or state in which the longitudinal axis 913 of the linear array transducer 18 of the acoustic source 12 is not parallel to the shaft 26 of the catheter 14 (e.g., the shaft axis 3500 is not parallel to the longitudinal axis 913). This misalignment can be corrected by rotating the linear array transducer 18 about its acoustic axis 3402, as described in the flowchart of FIG. 33. From the perspective view in FIG. 37, the acoustic source 12 can be rotated counterclockwise about the acoustic axis 3402 to transition the acoustic source 12 to an aligned state (e.g., as shown in FIG. 29 and/or FIG. 40 (below)).

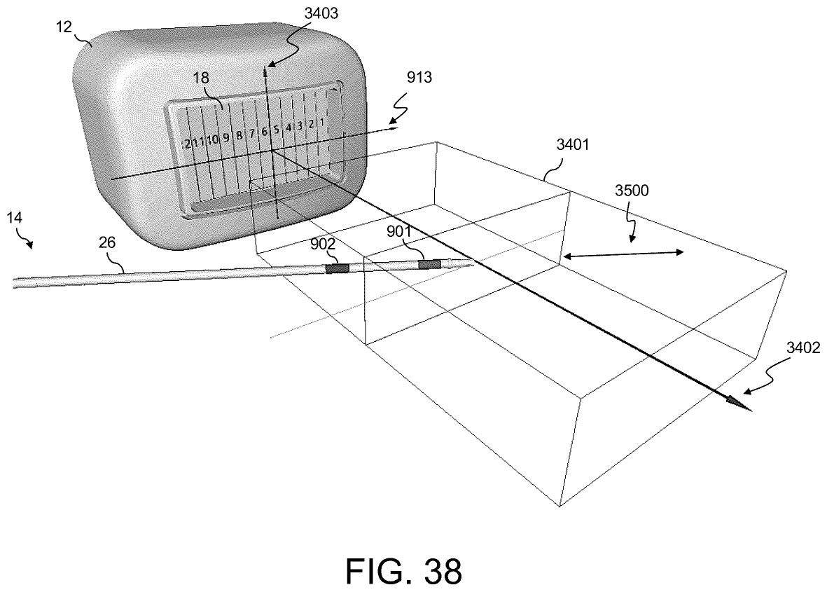

FIG. 38 is another front perspective view of the transducer 18 as seen from the direction of the catheter 14 according to one or more embodiments. FIG. 38 illustrates another rotational misalignment scenario or state in which the longitudinal axis 913 of the linear array transducer 18 of the acoustic source 12 is not parallel to the shaft 26 of the catheter 14 (e.g., the shaft axis 3500 is not parallel to the longitudinal axis 913), even though the catheter shaft 26 lies within the main symmetry plane of the linear array transducer 18 and/or the acoustic sensors 901 and 902 are located within the treatment region 3401. This misalignment can be corrected by rotating the acoustic source 12 about its lateral axis 3403 and shifting along the longitudinal axis 913, as described in the flowchart of FIG. 33. The longitudinal axis 913, the acoustic axis 3402, and the lateral axis 3403 are mutually orthogonal. In the perspective view in FIG. 38, the acoustic source 12 can be rotated counterclockwise about the positive direction of the lateral axis 3403 and translated in the positive direction of the longitudinal axis 913 to transition the acoustic source 12 into an aligned state (e.g., as shown in FIG. 29 and/or FIG. 40 (below)).

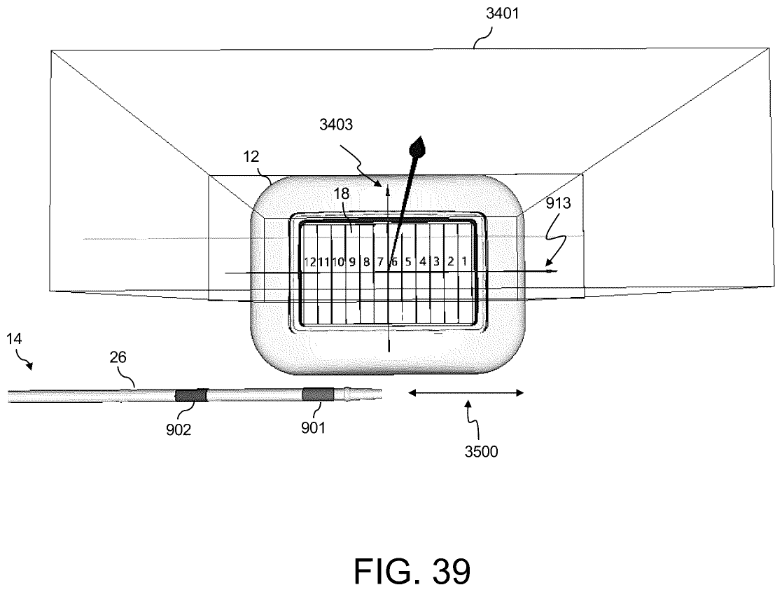

FIG. 39 is another front perspective view of the transducer 18 as seen from the direction of the catheter 14 according to one or more embodiments. FIG. 39 illustrates another possible misalignment scenario or state in which the catheter 14 is initially positioned laterally (e.g., with respect to the lateral axis 3403) outside of the main symmetry plane of the linear array transducer 18 of the acoustic source 12 and/or laterally (e.g., with respect to the lateral axis 3403) outside of the treatment region 3401. However, the shaft axis 3500 is parallel to the longitudinal axis 913 in FIG. 39. This misalignment can be corrected by rotating the linear array transducer 18 about its longitudinal axis 913 and/or by shifting (e.g., translating or moving) the acoustic source 12 along the lateral axis 3403 perpendicular to the longitudinal axis 913 of the linear array transducer 18, as described in the flowchart in FIG. 33. In the perspective view of FIG. 39, the acoustic source 12 can be rotated clockwise about the longitudinal axis 913 and/or shifted laterally downward, along the lateral axis 3403, towards the acoustic sensors 901, 902 (e.g., downward in FIG. 39). These movements bring the acoustic source 12 into an aligned state (e.g., as shown in FIG. 29 and/or FIG. 40 (below)).

FIG. 40 shows a front perspective view of the transducer 18 as seen from the direction of the catheter 14 according to one or more embodiments. In this example, the catheter sensors 901 and 902 are positioned in front of the acoustic source 12, depicted as a linear array of piezoelectric transducer elements 18. The acoustic source 12 is spatially aligned with the catheter 14 such that the longitudinal axis 913 of the linear array transducer 18 is parallel to the shaft 26 of the catheter 14 (e.g., the shaft 26 extends parallel to the longitudinal axis 913), and the tip of the catheter 14 is spatially aligned with the acoustic axis 3402 of the acoustic source 12. The shaft 26 extends along or parallel to a shaft axis 3500. The shaft axis 3500 is parallel to the longitudinal axis 913 in FIG. 40. This figure shows one example of a desirable alignment of the acoustic source with the catheter, in which the acoustic axis of the source 12 is aligned with (e.g., passes through) the tip of the catheter 14.

The invention should not be considered limited to the particular embodiments described above. Various modifications, equivalent processes, as well as numerous structures to which the invention may be applicable, will be readily apparent to those skilled in the art to which the invention is directed upon review of this disclosure. The above-described embodiments may be implemented in numerous ways. One or more aspects and embodiments involving the performance of processes or methods may utilize program instructions executable by a device (e.g., a computer, a processor, or other device) to perform, or control performance of, the processes or methods.

In this respect, various inventive concepts may be embodied as a non-transitory computer readable storage medium (or multiple non-transitory computer readable storage media) (e.g., a computer memory of any suitable type including transitory or non-transitory digital storage units, circuit configurations in Field Programmable Gate Arrays or other semiconductor devices, or other tangible computer storage medium) encoded with one or more programs that, when executed on one or more computers or other processors, perform methods that implement one or more of the various embodiments described above. When implemented in software (e.g., as an app), the software code may be executed on any suitable processor or collection of processors, whether provided in a single computer or distributed among multiple computers.

Further, it should be appreciated that a computer may be embodied in any of a number of forms, such as a rack-mounted computer, a desktop computer, a laptop computer, or a tablet computer, as non-limiting examples. Additionally, a computer may be embedded in a device not generally regarded as a computer but with suitable processing capabilities, including a Personal Digital Assistant (PDA), a smartphone or any other suitable portable or fixed electronic device.

Also, a computer may have one or more communication devices, which may be used to interconnect the computer to one or more other devices and/or systems, such as, for example, one or more networks in any suitable form, including a local area network or a wide area network, such as an enterprise network, and intelligent network (IN) or the Internet. Such networks may be based on any suitable technology and may operate according to any suitable protocol and may include wireless networks or wired networks.

Also, a computer may have one or more input devices and/or one or more output devices. These devices can be used, among other things, to present a user interface. Examples of output devices that may be used to provide a user interface include printers or display screens for visual presentation of output and speakers or other sound generating devices for audible presentation of output. Examples of input devices that may be used for a user interface include keyboards, and pointing devices, such as mice, touch pads, and digitizing tablets. As another example, a computer may receive input information through speech recognition or in other audible formats.

The non-transitory computer readable medium or media may be transportable, such that the program or programs stored thereon may be loaded onto one or more different computers or other processors to implement various one or more of the aspects described above. In some embodiments, computer readable media may be non-transitory media.

The terms “program,” “app,” and “software” are used herein in a generic sense to refer to any type of computer code or set of computer-executable instructions that may be employed to program a computer or other processor to implement various aspects as described above. Additionally, it should be appreciated that, according to one aspect, one or more computer programs that when executed perform methods of this application need not reside on a single computer or processor but may be distributed in a modular fashion among a number of different computers or processors to implement various aspects of this application.

Computer-executable instructions may be in many forms, such as program modules, executed by one or more computers or other devices. Generally, program modules include routines, programs, objects, components, data structures, etc. that performs particular tasks or implement particular abstract data types. The functionality of the program modules may be combined or distributed as desired in various embodiments.

Also, data structures may be stored in computer-readable media in any suitable form. For simplicity of illustration, data structures may be shown to have fields that are related through location in the data structure. Such relationships may likewise be achieved by assigning storage for the fields with locations in a computer-readable medium that convey relationship between the fields. However, any suitable mechanism may be used to establish a relationship between information in fields of a data structure, including through the use of pointers, tags or other mechanisms that establish relationship between data elements.

Thus, the disclosure and claims include new and novel improvements to existing methods and technologies, which were not previously known nor implemented to achieve the useful results described above. Users of the method and system will reap tangible benefits from the functions now made possible on account of the specific modifications described herein causing the effects in the system and its outputs to its users. It is expected that significantly improved operations can be achieved upon implementation of the claimed invention, using the technical components recited herein.

Also, as described, some aspects may be embodied as one or more methods. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

What is claimed is:

1. A method for localizing an acoustic source and a medical device with respect to each other, the method comprising:
a. introducing the medical device into a mammal, the medical device including an acoustic sensor;
b. acoustically coupling the acoustic source to the mammal at a position that corresponds to a target location of the medical device, the acoustic source comprising at least a first source transducer element and a second source transducer element;
c. sequentially producing acoustic signals with at least the first and second transducer elements;
d. receiving the acoustic signals with the acoustic sensor on or in the medical device, the acoustic sensor in electrical or wireless communication with a detector;
e. determining, with the detector, one-way time-of-flights (ToFs) of the acoustic signals transmitted at least from the first source transducer element to the acoustic sensor and from the second source transducer element to the acoustic sensor;
f. determining, with the detector, an alignment state of the acoustic source with respect to the medical device based, at least in part, on the one-way ToFs of the acoustic signals;
g. generating, with the detector, graphical feedback data indicating the alignment state of the acoustic source with respect to the medical device; and
h. displaying, on a display in communication with the detector, the graphical feedback data.

2. The method of claim 1, further comprising determining, with the detector and using the one-way ToFs of the acoustic signals, an axial position of the acoustic source relative to the medical device.

3. The method of claim 2, further comprising:
determining, with the detector, (a) a first distance between the first transducer element and the acoustic sensor using a first one-way ToF of the acoustic signals transmitted from the first source transducer element and the acoustic sensor (b) a second distance between the second transducer element and the acoustic sensor using a second one-way ToF of the acoustic signals transmitted from the second source transducer element and the acoustic sensor;
determining, with the detector, a radial distance between the acoustic source and the acoustic sensor using the first and second distances, the radial distance measured along a radial axis that is orthogonal to a reference axis that extends along a surface of the first and second source transducer elements,
wherein an intersection of the radial axis and the reference axis corresponds to the axial position of the acoustic source relative to the medical device.

4. The method of claim 2, wherein:
the alignment state is an axial misalignment of the acoustic source with respect to the medical device in a first direction along a reference axis that extends along a surface of the first and second source transducer elements, and
the graphical feedback data indicates a second direction to move the acoustic source along or parallel to the reference axis, the second direction opposite to the first direction.

5. The method of claim 4, further comprising:
i. moving the acoustic source along or parallel to the reference axis; and
j. repeating steps c-h in a loop so as to update the graphical feedback data on the display in real time while moving the acoustic source in step i.

6. The method of claim 5, further comprising:
determining, with the detector and using the one-way ToFs of the acoustic signals, that the acoustic source is axially aligned with the medical device; and
after determining that the acoustic source is axially aligned with the medical device:
changing the alignment state to an axial alignment of the acoustic source with respect to the medical device; and
updating the graphical feedback data to indicate the axial alignment of the acoustic source with respect to the medical device.

7. The method of claim 1, wherein the acoustic sensor is disposed closer to a distal end of the medical device than to a proximal end of the medical device, such that the alignment state corresponds to relative positions of the acoustic source and the distal end of the medical device.

8. A system comprising:
a medical device having a shaft configured to be inserted into a mammal, the shaft including an acoustic sensor;
an acoustic source including a housing and a transducer having a plurality of transducer elements including first and second source transducer elements, the acoustic source configured to be acoustically coupled to an external surface of the mammal;
a controller in communication with the acoustic source, the controller configured to cause the at least first and second source transducer elements to sequentially produce acoustic signals;
a detector in communication with the acoustic sensor, the detector including a processor and non-volatile memory coupled to the processor, the non-volatile memory storing computer-readable instructions that, when executed by the processor, cause the processor to:
a. determine one-way time-of-flights (ToFs) of the acoustic signals transmitted at least from the first source transducer element to the acoustic sensor and from the second source transducer element to the acoustic sensor;
b. determine an alignment state of the acoustic source with respect to the medical device based, at least in part, on the one-way ToFs of the acoustic signals; and
c. generate graphical feedback data indicating the alignment state of the acoustic source with respect to the medical device; and
a display in communication with the detector and configured to display the graphical feedback data.

9. The system of claim 8, wherein the computer-readable instructions, when executed by the processor, further cause the processor to determine, using the one-way ToFs of the acoustic signals, an axial position of the acoustic source relative to the medical device.

10. The system of claim 9, wherein the computer-readable instructions, when executed by the processor, further cause the processor to:
calculate (a) a first distance between the first transducer element and the acoustic sensor using a first one-way ToF of the acoustic signals transmitted from the first source transducer element and the acoustic sensor (b) a second distance between the second transducer element and the acoustic sensor using a second one-way ToF of the acoustic signals transmitted from the second source transducer element and the acoustic sensor; and
determine a radial distance between the acoustic source and the acoustic sensor using the first and second distances, the radial distance measured along a radial axis that is orthogonal to a reference axis that extends along a surface of the first and second source transducer elements,
wherein an intersection of the radial axis and the reference axis corresponds to the axial position of the acoustic source relative to the medical device.

11. The system of claim 8, wherein:
the alignment state is an axial misalignment of the acoustic source with respect to the medical device in a first direction along a reference axis that extends along a surface of the first and second source transducer elements, and
the graphical feedback data indicates a second direction to move the acoustic source along or parallel to the reference axis, the second direction opposite to the first direction.

12. The system of claim 8, wherein:
the acoustic sensor is a first acoustic sensor,
the shaft includes a second acoustic sensor that is axially displaced from the first acoustic sensor along the shaft,
the one-way ToFs are determined at least from the first source transducer element to each of the first and second acoustic sensors and from the second source transducer element to each of the first and second acoustic sensors, and
the computer-readable instructions, when executed by the processor, further cause the processor to:
determine an axial alignment state of the acoustic source relative to each of the first and second acoustic sensors, the axial alignment state determined with respect to a first reference axis that extends along a surface of the first and second source transducer elements;
determine a first rotational alignment state of the medical device relative to the acoustic source, the first rotational alignment state representing a rotational orientation of the acoustic source about the first reference axis; and
determine a second rotational alignment state of the acoustic source relative to the medical device, the second rotational alignment state representing a rotational orientation of the acoustic source about a second reference axis that is orthogonal to the first reference axis.

13. The system of claim 12, wherein the computer-readable instructions, when executed by the processor, further cause the processor to:
calculate, using the one-way ToFs of the acoustic signals sent from the first and second transducer elements to the first acoustic sensor, an axial position of the first acoustic sensor relative to the acoustic source;
calculate, using the one-way ToFs of the acoustic signals sent from the first and second transducer elements to the second acoustic sensor, an axial position of the second acoustic sensor relative to the acoustic source;
compare the axial positions of the first and second acoustic sensors relative to the acoustic source with a center of the transducer; and
determine that the first and second acoustic sensors are axially aligned with the transducer when the axial positions of the first and second acoustic sensors are within a predetermined range of the center of the transducer,
wherein the graphical feedback data indicates an axial alignment of the transducer relative to the first and second acoustic sensors when the axial positions of the first and second acoustic sensors are within the predetermined range of the center of the transducer.

14. The system of claim 13, wherein the computer-readable instructions, when executed by the processor, further cause the processor to:
determine that first acoustic sensor is not axially aligned with the transducer when the axial position of the first acoustic sensor is outside of the predetermined range of the center of the transducer; and
determine that second acoustic sensor is not axially aligned with the transducer when the axial position of the second acoustic sensor is outside of the predetermined range of the center of the transducer,
wherein the graphical feedback data indicates an axial misalignment of the transducer relative to the first acoustic sensor and/or the second acoustic sensor when the axial position of the first acoustic sensor is outside of the predetermined range of the center of the transducer and/or the axial position of the second acoustic sensor is outside of the predetermined range of the center of the transducer, respectively.

15. The system of claim 14, wherein the graphical feedback data further indicates an axial direction to move the acoustic source to achieve the axial alignment of the transducer relative to the first and second acoustic sensors.

16. The system of claim 14, wherein the computer-readable instructions, when executed by the processor, further cause the processor to:
calculate (a) a first distance between the first transducer element and the first acoustic sensor using a one-way ToF of the acoustic signals transmitted from the first source transducer element and the first acoustic sensor (b) a second distance between the second transducer element and the first acoustic sensor using a one-way ToF of the acoustic signals transmitted from the second source transducer element and the first acoustic sensor;
calculate (c) a third distance between the first transducer element and the second acoustic sensor using a one-way ToF of the acoustic signals transmitted from the first source transducer element and the second acoustic sensor (d) a fourth distance between the second transducer element and the first acoustic sensor using a second one-way ToF of the acoustic signals transmitted from the second source transducer element and the second acoustic sensor;
determine a first radial distance between the acoustic source and the first acoustic sensor using the first and second distances, the first radial distance measured along a first radial axis that is orthogonal to the first reference axis and that is parallel to the second reference axis; and
determine a second radial distance between the acoustic source and the second acoustic sensor using the third and fourth distances, the second radial distance measured along a second radial axis that is orthogonal to the first reference axis and that is parallel to the second reference axis,
wherein:
an intersection of the first radial axis and the first reference axis corresponds to a first axial position of the first acoustic sensor relative to the transducer,
an intersection of the second radial axis and the first reference axis corresponds to a second axial position of the first acoustic sensor relative to the transducer, and
the graphical feedback data includes a representation of the first and second axial positions.

17. The system of claim 16, wherein the computer-readable instructions, when executed by the processor, further cause the processor to:
compare the first and second radial distances;
determine that the transducer is rotationally aligned, about the first reference axis, with the first and second acoustic sensors when the first and second radial distances are within a predetermined range of each other; and
determine that the transducer is not rotationally aligned, about the first reference axis, with the first and second acoustic sensors when the first and second radial distances are outside of the predetermined range of each other,
wherein:
the graphical feedback data indicates a rotational alignment of the transducer, about the first reference axis, with respect to the first and second acoustic sensors when the first and second radial distances are within a predetermined range of each other, and
the graphical feedback data indicates a rotational misalignment of the transducer, about the first reference axis, with the first and second acoustic sensors when the first and second radial distances are outside of the predetermined range of each other.

18. The system of claim 16, wherein the graphical feedback data further indicates a rotational direction to rotate the acoustic source, about the first reference axis, to achieve the rotational alignment of the transducer, about the first reference axis, with respect to the first and second acoustic sensors.

19. The system of claim 16, wherein the computer-readable instructions, when executed by the processor, further cause the processor to:
compare the first and second axial positions;
determine that the transducer is rotationally aligned, about the second reference axis, with the first and second acoustic sensors when the first and second axial positions are within a predetermined range of each other; and
determine that the transducer is not rotationally aligned, about the second reference axis, with the first and second acoustic sensors when the first and second axial positions are outside of the predetermined range of each other,
wherein:
the graphical feedback data indicates that a rotational alignment of the transducer, about the second reference axis, with the first and second acoustic sensors when the first and second axial positions are within a predetermined range of each other, and
the graphical feedback data indicates a rotational misalignment of the transducer, about the second reference axis, with the first and second acoustic sensors when the first and second axial positions are outside of the predetermined range of each other.

20. The system of claim 19, wherein the graphical feedback data further indicates a rotational direction to rotate the acoustic source, about the second reference axis, to achieve the rotational alignment of the transducer, about the second reference axis, with respect to the first and second acoustic sensors.
